(19) United States
(12) Patent Application Publication
(10) Pub. No.: US 2009/0181920 A1
(43) Pub. Date: Jul. 16, 2009

(54) INTRATHECAL TREATMENT OF NEUROPATHIC PAIN WITH A2AR AGONISTS

(75) Inventors: Linda Watkins, Boulder, CO (US); Lisa C. Loram, Boulder, CO (US); Mark Hutchinson, Boulder, CO (US); Robert D. Thompson, Charlottesville, VA (US); Anthony Beauglehole, Charlottesville, VA (US); Frank W. Schmidtmann, Charlottesville, VA (US); Jayson M. Rieger, Charlottesville, VA (US)

Correspondence Address: CLINICAL DATA, INC., ATTN: MATTHEW CATLETT, ONE GATEWAY CENTER SUITE 702, NEWTON, MA, 02458, US

(73) Assignee: PGXHEALTH, LLC, Newton, MA (US)

(21) Appl. No.: 12/351,209

(22) Filed: Jan. 9, 2009

Related U.S. Application Data
(60) Provisional application No. 61/019,912, filed on Jan. 9, 2008.

(51) Int. Cl. A61K 31/7076; A61P 25/00

(52) U.S. Cl. 514/45

(57) ABSTRACT

The present invention relates to a method of treating neuropathic pain via intrathecal administration of agonists of A_{2A} adenosine receptors (ARs).

### CROSS-REFERENCE TO RELATED APPLICATIONS

The present application claims the priority benefits of U.S. Provisional Application No. 61/019,912, filed 9 Jan. 2008, which is expressly incorporated fully herein by reference.

### STATEMENT AS TO FEDERALLY FUNDED RESEARCH

This invention was partially funded by NIH Grants DA 015642 & DA017670 from the National Institute of Health. The government may have certain rights in the invention.

### FIELD OF THE INVENTION

The present invention relates to a method of treating neuropathic pain intrathecally using agonists of A_{2A} adenosine receptors (ARs).

### BACKGROUND OF THE INVENTION

Activated spinal cord microglia and astrocytes appear to contribute to the creation and maintenance of neuropathic pain. In particular, activated glia appear to do so, at least in part, via their release of the proinflammatory cytokines interleukin-1 (IL1), tumor necrosis factor (TNF), and IL6 (for review, see Watkins et al., Trends in Neurosci. (2001) 24:450-455). These proinflammatory cytokines amplify pain by enhancing the release of “pain” neurotransmitters from incoming sensory nerve terminals and by enhancing the excitability of spinal cord dorsal horn pain transmission neurons (Reeve et al., Eur. J. Pain (2000) 4:247-257; Watkins et al., Trends in Neurosci. (2001) 24:450-455).

Unfortunately, neuropathic pain remains a major unresolved problem, necessitating the identification of effective novel therapeutics. Adenosine is a neuromodulator regulating neuronal and non-neuronal cell function, and an immunomodulator acting as an anti-inflammatory agent on immune cells. Adenosine acts on four different subtypes of adenosine receptors, where agents selective for A_{2A}R (adenosine 2A receptors) in circulating immune cells decrease pro-inflammatory cytokine release and increase the potent anti-inflammatory cytokine, interleukin-10 (IL-10). Microglia within the spinal cord are the primary resident immune cells and are involved fundamentally in the induction and maintained production of mediators involved in chronic pain. Therefore, it was postulated that A_{2A}R agonists may be potentially potent therapeutic agents against neuropathic pain.

A wide variety of A_{2A} adenosine receptor agonists are now known in the art. These include U.S. Pat. No. 6,232,297 to Linden, et al. which describe compounds having the general formula:

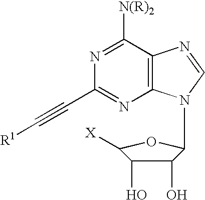

wherein each R can be H, X can be ethylaminocarbonyl and R¹ can be R¹ can be 4-methoxycarbonylcyclohexylmethyl (DWH-146e). These compounds are reported to be A_{2A} agonists.

U.S. Pat. No. 7,214,665 to Linden, et al. describes compounds having the general formula:

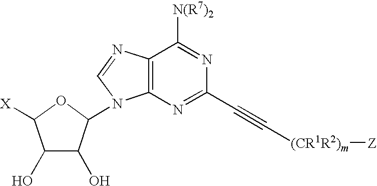

wherein R⁷ can be H, X can be an ether or an amide, CR¹R² can be CH₂, and Z can be a heterocyclic ring. These compounds are reported to be A_{2A} agonists.

U.S. Pat. Appl. No. 2006/004088 to Rieger, et al. describes compounds having the general formula:

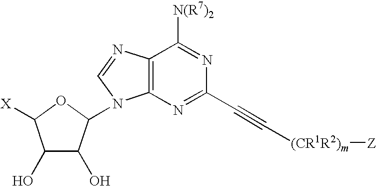

wherein R⁷ can be H, X can be a cycloalkyl-substituted ether or amide, CR¹R² can be CH₂, and Z can be a heterocyclic ring. These compounds are reported to be A_{2A} agonists.

U.S. Pat. Appl. No. 2007/0270373 to Rieger, et al. describes compounds having the general formula:

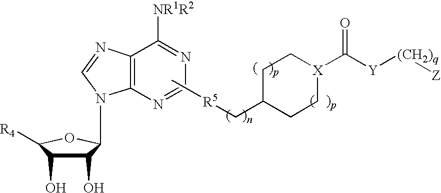

wherein NR¹R² can be NH₂, R⁴ can be a an ether or an amide, R⁵ can be ethynyl, Y can be O or NR¹, and Z can be an aryl or heteroaryl. These compounds are reported to be A_{2A} agonists.

G. Cristalli (U.S. Pat. No. 5,593,975) describe 2-arylethynyl, 2-cycloalkylethynyl or 2-hydroxyalkylethynyl derivatives, wherein the riboside residue is substituted by carboxy amino, or substituted carboxy amino. These compounds are reported to be A_{2A} agonists.

In view of the above, it is desirable to find new methods of treating neuropathic pain.

### SUMMARY OF THE INVENTION

The present invention provides a therapeutic method for treating neuropathic pain, comprising intrathecally administering to a patient in need thereof a therapeutically effective amount of an A_{2A} adenosine receptor agonist.

The present invention also provides a pharmaceutical composition useful for treating neuropathic pain (e.g., a composition suitable for intrathecal administration), comprising an effective amount of an A_{2A} adenosine receptor agonist and a pharmaceutically acceptable excipient.

The present invention also provides compounds of the invention for use in medical therapy.

The present invention also provides the use of a compound of the present invention for the manufacture of a medicament for treating neuropathic pain.

These and other aspects of the present invention have been accomplished by the discovery that neuropathic pain can be treated by intrathecal administration of A_{2A} agonists.

### BRIEF DESCRIPTION OF THE FIGURES

FIG. 1 illustrates the effects of CGS21680 (3-[4-[2-[[6-amino-9-[(2R,3R,4S,5S)-5-(ethylcarbamoyl)-3,4-dihydroxy-oxolan-2-yl]purin-2-yl]amino]ethyl]phenyl]propanoic acid) and ATL313 (4-{3-[6-amino-9-(5-cyclopropylcarbamoyl-3,4-dihydroxy-tetrahydro-furan-2-yl)-9H-purin-2-yl]-prop-2-ynyl}-piperidine-1-carboxylic acid methyl ester) in rats using the unilateral chronic constriction injury (CCI) pain model.

FIG. 2 illustrates the effects of simultaneous (top panel) co-administration of ATL313 and ZM241385 (an A_{2A} antagonist)(4-(2-[7-amino-2-(2-furyl[1,2,4]-triazolo{2,3-α[1,3,5]triazin-5-yl-aminoethyl)phenol), 10-14 days after CCI surgery and the co-administration wherein ZM241385 was administered one week following ATL313 administration.

FIG. 3 illustrates the effects of CGS21680, ATL313, Compound A (4-{3-[6-Amino-9-(5-cyclopropylcarbamoyl-3,4-dihydroxy-tetrahydro-furan-2-yl)-9H-purin-2-yl]-prop-2-ynyl}-piperidine-1-carboxylic acid 2-methoxyphenyl ester), Compound B (4-{3-[6-Amino-9-(5-cyclopropylcarbamoyl-3,4-dihydroxy-tetrahydro-furan-2-yl)-9H-purin-2-yl]-prop-2-ynyl}-piperidine-1-carboxylic acid cyclobutyl ester), and Compound C (4-{3-[6-Amino-9-(5-cyclopropylcarbamoyl-3,4-dihydroxy-tetrahydro-furan-2-yl)-9H-purin-2-yl]-prop-2-ynyl}-piperidine-1-carboxylic acid cyclopropylmethyl ester) using the CCI pain model.

### DETAILED DESCRIPTION OF THE INVENTION

In an embodiment, the present invention provides a novel therapeutic method for treating neuropathic pain, comprising intrathecally administering to a patient in need thereof a therapeutically effective amount of an A_{2A} adenosine receptor agonist. It has surprisingly been discovered that the effect of A_{2A} agonists on neuropathic pain can have a very long duration. Thus, it may be desirable to administer the agonist in a daily, weekly (e.g., 1, 2, 3, 4, 5, or 6 weeks between administrations), biweekly, monthly, or even bimonthly regimen.

Examples of agonists of A_{2A} adenosine receptors that are expected to useful in the practice of the present invention include compounds having the formula I or a stereoisomer or pharmaceutically acceptable salt thereof:

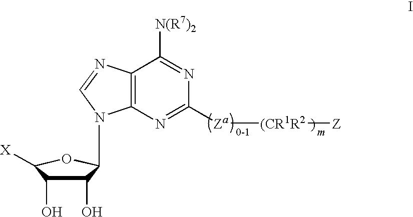

wherein

Z^{a} is C≡C, O, NH, or NHN═CR^{3a};

Z is CR³R⁴R⁵ or NR⁴R⁵;

each R¹ is independently hydrogen, halo, —OR^{a}, —SR^{a}, (C₁-C₈)alkyl, cyano, nitro, trifluoromethyl, trifluoromethoxy, (C₃-C₈)cycloalkyl, heterocycle, heterocycle(C₁-C₈)alkylene-, aryl, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b}), R^{b}R^{c}NC(═O)N(R^{b})—, R^{b}R^{c}NC(═S)N(R^{b})—, —OPO₃R^{a}, R^{a}OC(═S)—, R^{a}C(═S)—, —SSR^{a}, R^{a}S(═O)—, R^{a}S(═O)₂—, or —N═NR^{b};

each R² is independently hydrogen, halo, (C₁-C₈)alkyl, (C₃-C₈)cycloalkyl, heterocycle, heterocycle(C₁-C₈)alkylene-, aryl, aryl(C₁-C₈)alkylene-, heteroaryl, or heteroaryl(C₁-C₈)alkylene-;

alternatively, R¹ and R² and the atom to which they are attached is C═O, C═S or C═NR^{d},

R⁴ and R⁵ are independently H or (C₁-C₈)alkyl;

alternatively, R⁴ and R⁵ together with the atom to which they are attached form a saturated, partially unsaturated, or aromatic ring that is mono-, bi- or polycyclic and has 3, 4, 5, 6, 7, 8, 9 or 10 ring atoms optionally having 1, 2, 3, or 4 heteroatoms selected from non-peroxide oxy (—O—), thio (—S—), sulfinyl (—SO—), sulfonyl (—S(O)₂—) or amine (—NR^{b}—) in the ring;

wherein R⁴ and R⁵ are independently substituted with 0-3 R⁶ groups or any ring comprising R⁴ and R⁵ is substituted with from 0 to 6 R⁶ groups;

each R⁶ is independently hydrogen, halo, —OR^{a}, —SR^{a}, (C₁-C₈)alkyl, cyano, nitro, trifluoromethyl, trifluoromethoxy, (C₁-C₈)cycloalkyl, (C₆-C₁₂)bicycloalkyl, heterocycle, heterocycle (C₁-C₈)alkylene-, aryl, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, —OPO₃R^{a}, R^{a}OC(═S)—, R^{a}C(═S)—, —SSR^{a}, R^{a}S(═O)—, —NNR^{b}, or two R⁶ groups and the atom to which they are attached is C═O, C═S; or two R⁶ groups together with the atom or atoms to which they are attached can form a carbocyclic or heterocyclic ring comprising from 1-6 carbon atoms and 1, 2, 3, or 4 heteroatoms selected from non-peroxide oxy (—O—), thio (—S—), sulfinyl (—SO—), sulfonyl (—S(O)₂—) or amine (—NR^{b}—) in the ring;

R³ is hydrogen, halo, —OR^{a}, —SR^{a}, (C₁-C₈)alkyl, cyano, nitro, trifluoromethyl, trifluoromethoxy, (C₃-C₈)cycloalkyl, heterocycle, heterocycle(C₁-C₈)alkylene-, aryl, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, —OPO₃R^{a}, R^{a}OC(═S)—, R^{a}C(═S)—, —SSR^{a}, R^{a}S(═O)—, R^{a}S(═O)₂—, —NNR^{b}; or if the ring formed from CR⁴R⁵ is aryl or heteroaryl or partially unsaturated then R³ can be absent;

R^{3a} is hydrogen, (C₁-C₈)alkyl, or aryl;

each R⁷ is independently hydrogen, (C₁-C₈)alkyl, (C₃-C₈)cycloalkyl, aryl, aryl(C₁-C₈)alkylene, heteroaryl, or heteroaryl(C₁-C₈)alkylene-;

X is —CH₂OR^{a}, —CO₂R^{a}, —CH₂OC(O)R^{a}, —C(O)NR^{b}R^{c}, —CH₂SR^{a}, —C(S)OR^{a}, —CH₂OC(S)R^{a}, —C(S)NR^{b}R^{c}, or —CH₂N(R^{b})(R^{c});

alternatively, X is an aromatic ring of the formula:

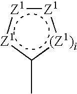

each Z¹ is non-peroxide oxy (—O—), S(O)₀₋₂, —C(R⁸)—, or amine (—NR⁸—), provided that at least one Z¹ is non-peroxide oxy (—O—), thio (—S—), sulfinyl (—SO—), sulfonyl (—S(O)₂—) or amine (—NR⁸—);

each R⁸ is independently hydrogen, (C₁-C₈)alkyl, (C₁-C₈)alkenyl, (C₃-C₈)cycloalkyl, (C₃-C₈)cycloalkyl(C₁-C₈)alkylene, (C₃-C₈)cycloalkenyl, (C₃-C₈)cycloalkenyl(C₁-C₈)alkylene, aryl, aryl(C₁-C₈)alkylene, heteroaryl, or heteroaryl(C₁-C₈)alkylene, wherein any of the alkyl or alkenyl groups of R⁸ are optionally interrupted by —O—, —S—, or —N(R^{a})—;

wherein any of the alkyl, cycloalkyl, heterocycle, aryl, or heteroaryl, groups of R¹, R², R³, R^{3a}, R⁶, R⁷ and R⁸ is optionally substituted on carbon with one or more (e.g. 1, 2, 3, or 4) substituents selected from the group consisting of halo, —OR^{a}, —SR^{a}, (C₁-C₈)alkyl, cyano, nitro, trifluoromethyl, trifluoromethoxy, (C₃-C₈)cycloalkyl, (C₆-C₁₂)bicycloalkyl, heterocycle, heterocycle(C₁-C₈)alkylene-, aryl, aryloxy, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, —OPO₃R^{a}, R^{a}OC(═S)—, R^{a}C(═S)—, —SSR^{a}, R^{a}S(═O)ₚ—, R^{b}R^{c}NS(O)ₚ—, and —N═NR^{b};

wherein any (C₁-C₈)alkyl, (C₃-C₈)cycloalkyl, (C₆-C₁₂)bicycloalkyl, (C₁-C₈)alkoxy, (C₁-C₈)alkanoyl, (C₁-C₈)alkylene, or heterocycle, is optionally partially unsaturated;

each R^{a}, R^{b} and R^{c} is independently hydrogen, (C₁-C₁₂)alkyl, (C₁-C₈)alkoxy, (C₁-C₈)alkoxy-(C₁-C₁₂)alkylene, (C₃-C₈)cycloalkyl, (C₃-C₈)cycloalkyl-(C₁-C₁₂)alkylene, (C₁-C₈)alkylthio, amino acid, aryl, aryl(C₁-C₈)alkylene, heterocycle, heterocycle-(C₁-C₈)alkylene, heteroaryl, or heteroaryl(C₁-C₈)alkylene;

alternatively R^{b} and R^{c}, together with the nitrogen to which they are attached, form a pyrrolidino, piperidino, morpholino, or thiomorpholino ring;

wherein any of the alkyl, cycloalkyl, heterocycle, aryl, or heteroaryl groups of R^{a}, R^{b} and R^{c} is optionally substituted on carbon with 1 or 2 substituents selected from the group consisting of halo, —(CH₂)₃OR^{e}, (CH₂)₃SR^{e}, (C₁-C₈)alkyl, (CH₂)ₐCN, (CH₂)ₐNO₂, trifluoromethyl, trifluoromethoxy, —(CH₂)ₐCO₂R³, (CH₂)ₐNR^{e}R^{e}, and (CH₂)ₐC(O)NR^{e}R^{e};

R^{d} is hydrogen or (C₁-C₆)alkyl;

R^{e} is independently selected from H and (C₁-C₆)alkyl;

a is 0, 1, or 2;

i is 1 or 2

m is 0 to 8; and

p is 0 to 2;

provided that m is at least 1 when Z is NR⁴R⁵; or

a pharmaceutically acceptable salt thereof.

Specific values listed below for radicals, substituents, and ranges, are for illustration only; they do not exclude other defined values or other values within defined ranges for the radicals and substituents.

For example, specific values include compounds having the formula (Ia):

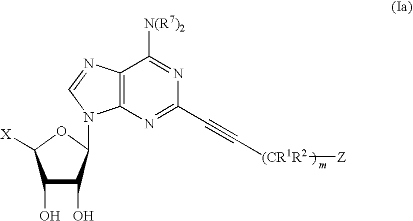

wherein

R¹ is hydrogen, —OH, —CH₂OH, —OMe, —OAc, —NH₂, —NHMe, —NMe₂ or —NHAc;

R² is hydrogen, (C₁-C₈)alkyl, cyclopropyl, cyclohexyl or benzyl;

R³ is hydrogen, OH, OMe, OAc, NH₂, NHMe, NMe₂ or NHAc;

CR⁴R⁵ or NR⁴R⁵ is optionally substituted with 0-2 R⁶ groups and is cyclopentane, cyclohexane, piperidine, dihydro-pyridine, tetrahydro-pyridine, pyridine, piperazine, tetrahydro-pyrazine, dihydro-pyrazine, pyrazine, dihydro-pyrimidine, tetrahydro-pyrimidine, hexahydro-pyrimidine, pyrazine, imidazole, dihydro-imidazole, imidazolidine, pyrazole, dihydro-pyrazole, and. pyrazolidine;

alternatively, the ring CR⁴R⁵ or NR⁴R⁵ is optionally substituted with 0-4 (e.g., 0 to 2) R⁶ groups and is selected from the group consisting of:

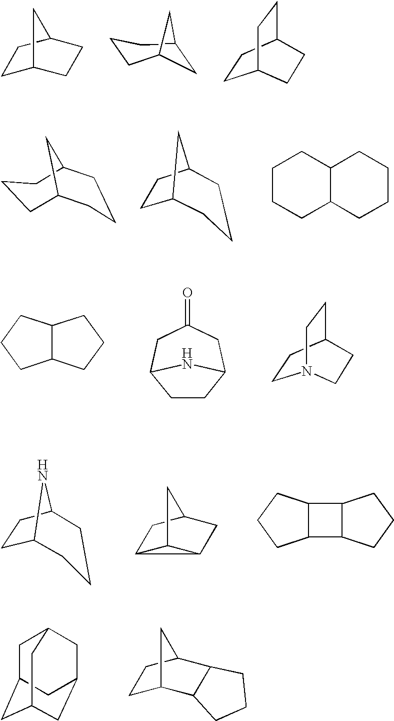

R⁶ is hydrogen, (C₁-C₈)alkyl, —OR^{a}, —CO₂R^{a}, R^{a}C(═O)—, R^{a}C(═O)O—, R^{b}R^{c}N—, R^{b}R^{c}NC(═O)—, or aryl;

R^{a}, R^{b} and R^{c} are independently hydrogen, (C₃-C₄)-cycloalkyl, (C₁-C₈)alkyl, aryl or aryl(C₁-C₈)alkylene;

each R⁷ is independently hydrogen, alkyl (e.g., C₁-C₈alkyl), aryl, aryl(C₁-C₈)alkylene or heteroaryl(C₁-C₈)alkylene;

R⁸ is methyl, ethyl, propyl, 2-propenyl, cyclopropyl, cyclobutyl, cyclopropylmethyl, —(CH₂)₂CO₂CH₃, or —(CH₂)₂₋₃OH;

X is —CH₂OR^{a}, —CO₂R^{a}, —CH₂OC(O)R^{a}, or —C(O)NR^{b}R^{c};

alternatively X is selected from:

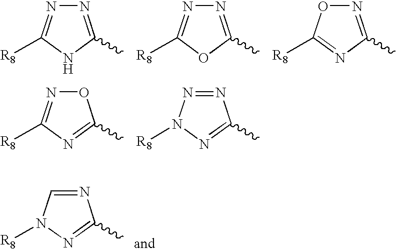

m is 0, 1 or 2;

or a pharmaceutically acceptable salt thereof.

Additional specific values include compounds having the formula (Ia), wherein:

R¹ is hydrogen, OH, OMe, or NH₂;

R² is hydrogen, methyl, ethyl or propyl;

R³ is hydrogen, OH, OMe, or NH₂; the ring CR⁴R⁵ or NR⁴R⁵ is selected from the group consisting of:

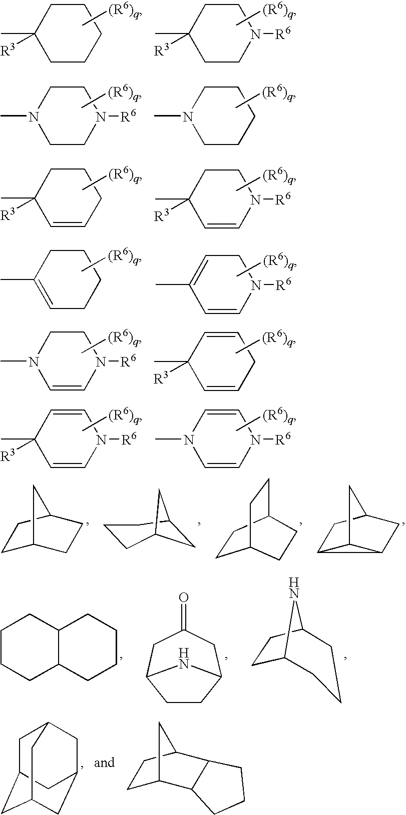

where q is from 0 to 4 (e.g., 0-2);

R⁶ is hydrogen, (C₁-C₈)alkyl, —OR^{a}, —CO₂R^{a}, R^{a}C(═O)—, R^{a}C(═O)O—, R^{b}R^{c}N—, R^{b}R^{c}NC(═O)—, or aryl;

R^{a} and R^{b} are independently hydrogen, methyl, ethyl, propyl, butyl, ethylhexyl, cyclopropyl, cyclobutyl, phenyl or benzyl;

N(R⁷)₂ is amino, methylamino, dimethylamino; ethylamino; pentylamino, diphenylethylamino, (pyridinylmethyl)amino, (pyridinyl)(methyl)amino, diethylamino or benzylamino; and,

R⁸ is methyl, ethyl, propyl, or cyclopropyl;

X is —CH₂OR^{a} or —C(O)NR^{b}R^{c};

alternatively, X is selected from:

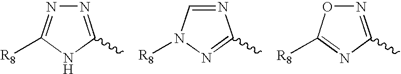

or a pharmaceutically acceptable salt thereof.

Additional specific values include compounds having the formula (Ia), wherein:

R¹ is hydrogen, OH, or NH₂;

R² is hydrogen or methyl;

R³ is hydrogen, OH, or NH₂;

the ring CR⁴R⁵ or NR⁴R⁵ is selected from the group consisting of:

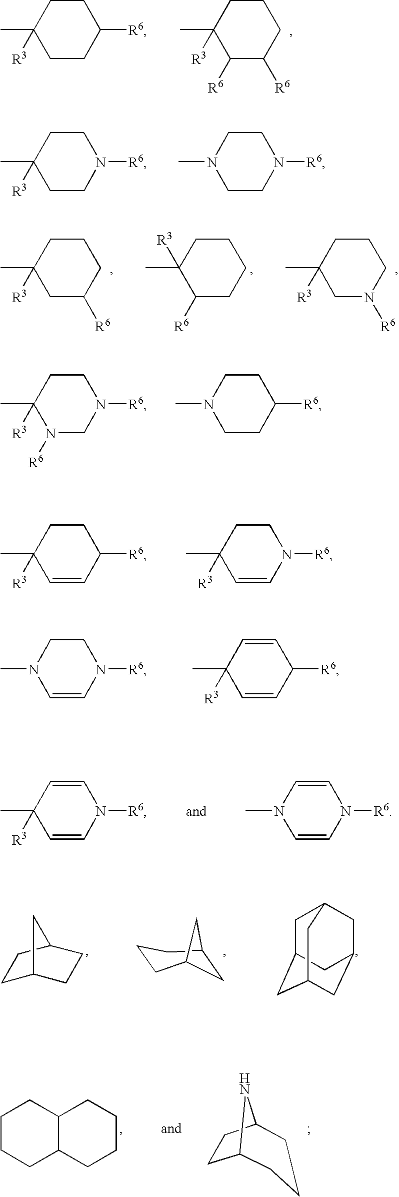

where q is from 0 to 2;

R⁶ is hydrogen, methyl, ethyl, t-butyl, phenyl, —CO₂R^{a}—CONR^{b}R^{c}, or R^{a}C(═O)—;

R^{b} is H;

R^{a} is methyl, ethyl, propyl, butyl, pentyl, ethylhexyl cyclopropyl, and cyclobutyl;

—N(R⁷)₂ is amino, methylamino, dimethylamino; ethylamino; diethylamino or benzylamino;

or a pharmaceutically acceptable salt thereof.

Additional specific values include compounds having the formula (Ia), wherein:

R¹ is hydrogen or OH;

R² is hydrogen;

R³ is hydrogen or OH;

the ring CR⁴R⁵ or NR⁴R⁵ is selected from the group consisting of:

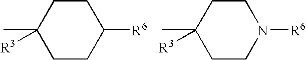

R⁶ is hydrogen, methyl, ethyl, —CO₂R³, and —CONR^{b}R^{c};

R^{b} is H;
R^{a} is methyl, ethyl, i-propyl, i-butyl, tert-butyl, and cyclopropyl;
N(R⁷)₂ is amino, or methylamino;
X is —CH₂OH,

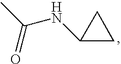

C(O)NHCH₃, or —C(O)NHCH₂CH₃;
or a pharmaceutically acceptable salt thereof.

Additional specific values include compounds wherein: the ring comprising R⁴, R⁵ and the atom to which they are connected is 2-methyl cyclohexane, 2,2-dimethylcyclohexane, 2-phenylcyclohexane, 2-ethylcyclohexane, 2,2-diethylcyclohexane, 2-tert-butyl cyclohexane, 3-methyl cyclohexane, 3,3-dimethylcyclohexane, 4-methyl cyclohexane, 4-ethylcyclohexane, 4-phenyl cyclohexane, 4-tert-butyl cyclohexane, 4-carboxymethyl cyclohexane, 4-carboxyethyl cyclohexane, 3,3,5,5-tetramethyl cyclohexane, 2,4-dimethyl cyclopentane, 4-cyclohexanecarboxylic acid, 4-cyclohexanecarboxylic acid esters, 4-methyloxyalkanoyl-cyclohexane, 4-piperidine-1-carboxylic acid methyl ester, 4-piperidine-1-carboxylic acid tert-butyl ester 4-piperidine, 4-piperazine-1-carboxylic acid methyl ester, 4-piperidine-1-carboxylic acid tert-butylester, 1-piperidine-4-carboxylic acid methyl ester, 1-piperidine-4-carboxylic acid tert-butyl ester, tert-butylester, 1-piperidine-4-carboxylic acid methyl ester, or 1-piperidine-4-carboxylic acid tert-butyl ester, 3-piperidine-1-carboxylic acid methyl ester, 3-piperidine-1-carboxylic acid tert-butyl ester, 3-piperidine, 3-piperazine-1-carboxylic acid methyl ester, 3-piperidine-1-carboxylic acid tert-butylester, 1-piperidine-3-carboxylic acid methyl ester, or 1-piperidine-3-carboxylic acid tert-butyl ester; or a pharmaceutically acceptable salt thereof.

Additional specific values include compounds having the formula (Ia), wherein:

R¹ is hydrogen or OH;

R² is hydrogen;

R³ is hydrogen or OH;

the ring CR⁴R⁵ or NR⁴R⁵ is selected from the group consisting of:

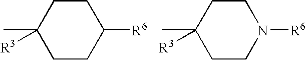

R⁶ is —CO₂R^{a};

R^{a} is (C₁-C₈)alkoxy, (C₃-C₆)cycloalkyl, (C₃-C₆)cycloalkyl-(C₁-C₃)alkylene, heterocycle, and heterocycle-(C₁-C₃)alkylene;

wherein any of the alkyl, cycloalkyl, heterocycle, aryl, or heteroaryl groups of R^{a}, R^{b} and R^{c} is optionally substituted on carbon with 1 or 2 substituents selected from the group consisting of halo, OR^{e}, (C₁-C₄)alkyl, —CN, NO₂, trifluoromethyl, trifluoromethoxy, CO₂R³, NR^{e}R^{e}, and C(O)NR^{e}R^{e}; and,

R^{e} is independently selected from H and (C₁-C₄)alkyl.

Exemplary compounds from that are expected to be useful in the present invention are shown in Table A below.

TABLE A

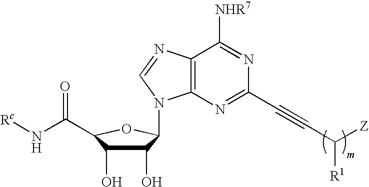

| Ex. # | R^{c} | R⁷ | —(R¹)ₘ—Z |
|---|---|---|---|
| 1. | Et | H | 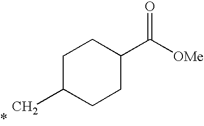 |
| 2. | Et | H | 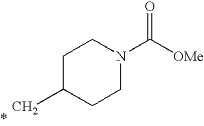 |
| 3. | cPr | H | 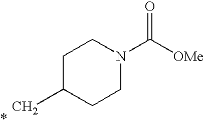 |
| 4. | Et | H | 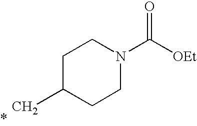 |
| 5. | cPr | H | 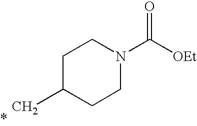 |
| 6. | Et | H | 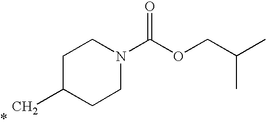 |
| 7. | cPr | H | 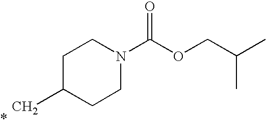 |
| 8. | Et | H | 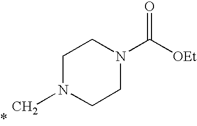 |
| 9. | Et | H | 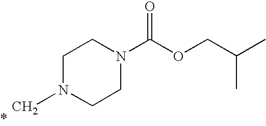 |
| 10. | Et | H | 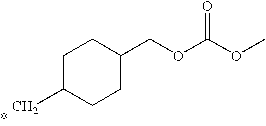 |
| 11. | Et | H | 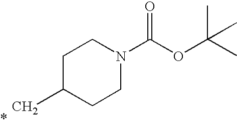 |
| 12. | cPr | H | 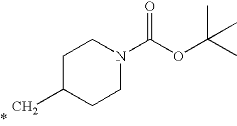 |
| 13. | Et | H | 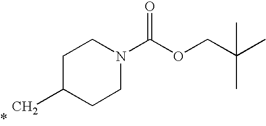 |
| 14. | cPr | H | 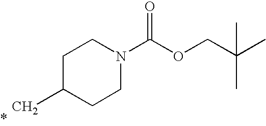 |
| 15. | Et | H | 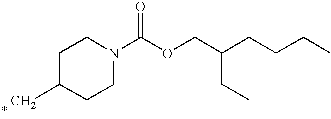 |
| 16. | cPr | H | 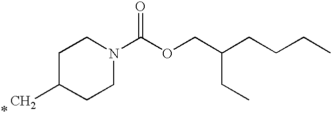 |
| 17. | cPr | H | 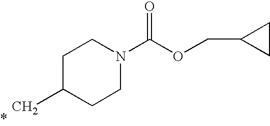 |
| 18. | Et | H | 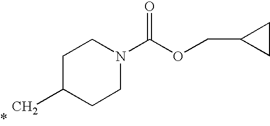 |
| 19. | cPr | H | 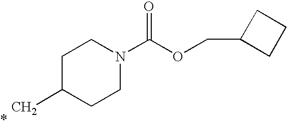 |
| 20. | Et | H | 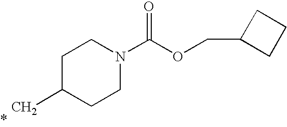 |
| 21. | cPr | H | 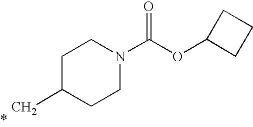 |
| 22. | Et | H | 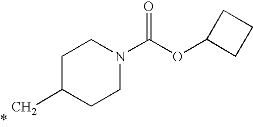 |
| 23. | Et | H | 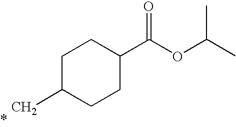 |
| 24. | cPr | H | 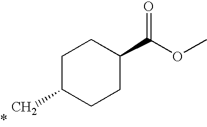 |
| 25. | Et | H | 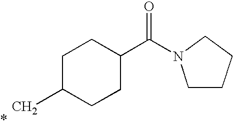 |
| 26. | Et | H | 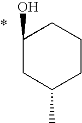 |
| 27. | Et | H | 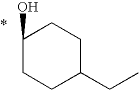 |
| 28. | Et | H | 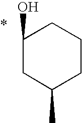 |
| 29. | Et | H | 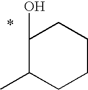 |
| 30. | Et | H | 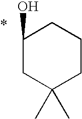 |
| 31. | cPr | H | 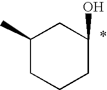 |
| 32. | Et | H | 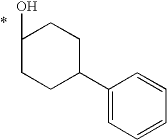 |
| 33. | Et | H | 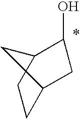 |
| 34. | cPr | H | 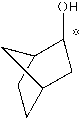 |
| 35. | cPr | H | 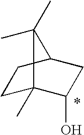 |
| 36. | Et | H | 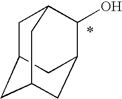 |
| 37. | cPr | H | 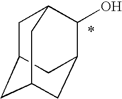 |
| 38. | Et | H | 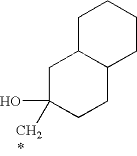 |
| 39. | cPr | H | 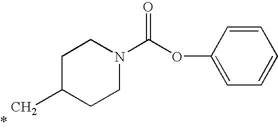 |
| 40. | Et | H | 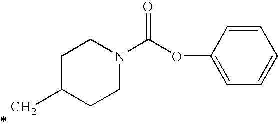 |
| 41. | cPr | H | 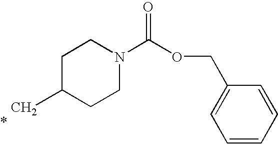 |
| 42. | Et | H | 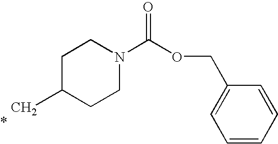 |

* signifies the point of attachment.

Further examples of agonists of A_{2A} adenosine receptors that are expected to useful in the practice of the present invention include compounds having the formula TI or a stereoisomer or pharmaceutically acceptable salt thereof:

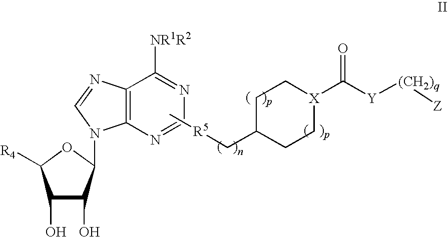

wherein:

R¹ and R² independently are selected from the group consisting of H, (C₁-C₈)alkyl, (C₃-C₈)cycloalkyl, (C₃-C₈)cycloalkyl(C₁-C₈)alkylene, aryl, aryl(C₁-C₈)alkylene, heteroaryl, heteroaryl(C₁-C₈)alkylene-, diaryl(C₁-C₈)alkylene, and diheteroaryl(C₁-C₈)alkylene, wherein the aryl and heteroaryl rings are optionally substituted with 1-4 groups independently selected from fluoro, chloro, iodo, bromo, methyl, trifluoromethyl, and methoxy;

each R independently is selected from the group consisting of H, C₁-C₄ alkyl, cyclopropyl, cyclobutyl, and (CH₂)ₐcyclopropyl;

X is CH or N, provided that when X is CH then Z cannot be substituted with halogen, C₁-C₆ alkyl, hydroxyl, amino, or mono- or di-(C₁-C₆-alkyl)amino;

Y is selected from the group consisting of O, NR¹, —(OCH₂CH₂O)ₘCH₂—, and —(NR¹CH₂CH₂O)ₘCH₂—, provided that when Y is O or NR¹, then at least one substituent is present on Z;

Z is selected from the group consisting of 5-membered heteroaryl, 6-membered aryl, 6-membered heteroaryl, carbocyclic biaryl, and heterocyclic biaryl, wherein the point of attachment of Y to Z is a carbon atom on Z, wherein Z is substituted with 0-4 groups independently selected from the group consisting of F, Cl, Br, I, (C₁-C₄)alkyl, —(CH₂)ₐOR³, —(CH₂)ₐNR³R³, —NHOH, —NR³NR³R³, nitro, —(CH₂)ₐCN, —(CH₂)ₐCO₂R³, —(CH₂)ₐCONR³R³, trifluoromethyl, and trifluoromethoxy;

alternatively, Y and Z together form an indolyl, indolinyl, isoindolinyl, tetrahydroisoquinolinyl, or tetrahydroquinolinyl moiety wherein the point of attachment is via the ring nitrogen and wherein said indolyl, indolinyl, isoindolinyl, tetrahydroisoquinolinyl, or tetrahydroquinolinyl moiety, which is substituted with 0-4 groups independently selected from the group consisting of F, Cl, Br, I, C₁-C₄ alkyl, —(CH₂)ₐOR³, —(CH₂)ₐNR³R³, —NHOH, —NR³NR³R³NO₂, —(CH₂)ₐCN, —(CH₂)ₐCO₂R³, —(CH₂)CONR³R³, CF₃, and OCF₃;

R³ is independently selected from the group consisting of H, (C₁-C₆)alkyl, cycloalkyl, aryl, and heteroaryl;

R⁴ is selected from the group consisting of CH₂OR, C(O)NRR, and CO₂R;

R⁵ is selected from the group consisting of CH₂CH₂, CH═CH, and C≡C;

a is selected from 0, 1, and 2;

m is selected from 1, 2, and 3;

n is selected from 0, 1, and 2;

each p independently is selected from 0, 1, and 2; and,

q is selected from 0, 1, and 2.

Additional specific values include compounds having the formula IIa or a pharmaceutically acceptable salt thereof:

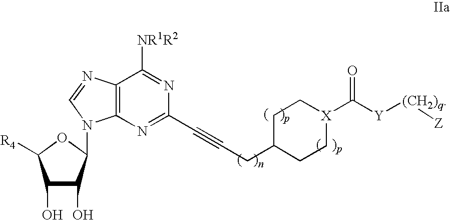

Additional specific values include compounds having the formula IIb or a pharmaceutically acceptable salt thereof:

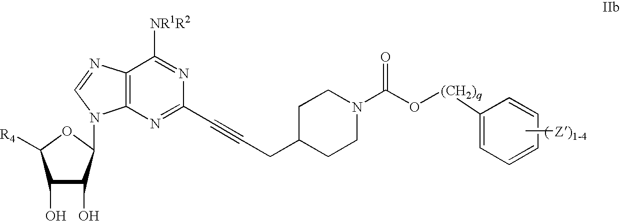

wherein:

each Z′ is independently selected from the group consisting F, Cl, Br, I, C₁-C₄ alkyl, —(CH₂)ₐOR³, —(CH₂)ₐNR³R³, —NHOH, —NR³NR³R³, NO₂, —(CH₂)ₐCN, —(CH₂)ₐCO₂R³, —(CH₂)ₐCONR³R³CF₃, and OCF₃.

Additional specific values include compounds wherein R is selected from H, methyl, ethyl or cyclopropyl.

Additional specific values include compounds having the formula IIc or a pharmaceutically acceptable salt thereof:

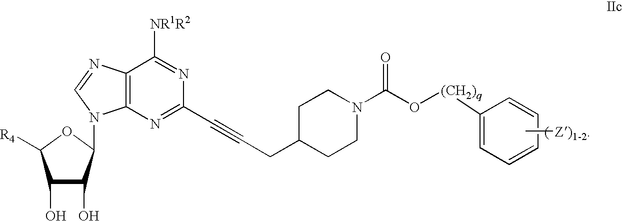

Additional specific values include compounds wherein Z′ is selected from the group consisting of F, Cl, methyl, OR³, NO₂, CN, NR³R³ and CO₂R³.

Additional specific values include compounds wherein R³ is methyl or hydrogen.

Additional exemplary compounds that are expected to be useful in the present invention are shown in Table B below.

TABLE B

| | |
|---|---|
| | i |
| 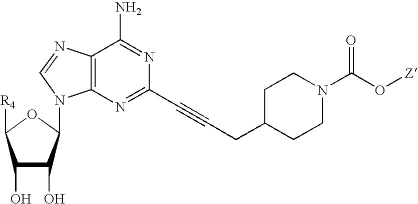 | |

| | |
|---|---|
| | ii |
| 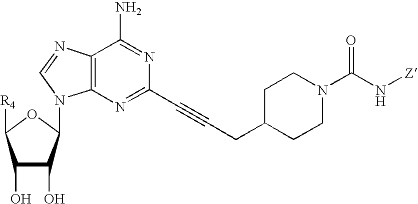 | |
| | iii |
| 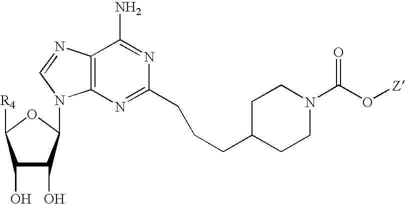 | |

| Ex. # | R⁴ | Z′ |
|---|---|---|
| 1 | C | 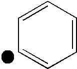 |
| 2 | C | 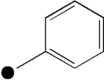 |
| 3 | C | 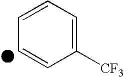 |
| 4 | A | 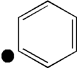 |
| 5 | C | 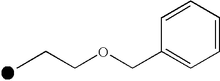 |
| 6 | A | 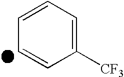 |
| 7 | A | 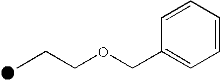 |
| 8 | C | 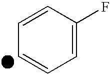 |
| 9 | C | 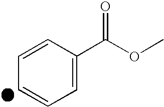 |
| 10 | C | 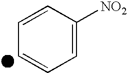 |
| 11 | A | 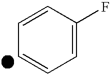 |
| 12 | A | 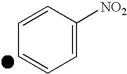 |
| 13 | A | 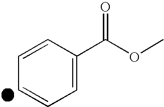 |
| 14 | C | 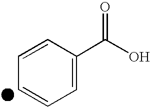 |
| 15 | B | 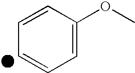 |
| 16 | B | 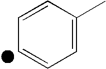 |
| 17 | C | 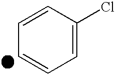 |
| 18 | C | 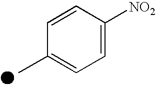 |
| 19 | B | 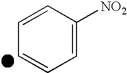 |
| 20 | C | 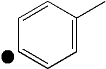 |
| 21 | C | 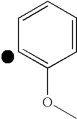 |
| 22 | C | 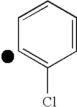 |
| 23 | C | 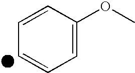 |
| 24 | B | 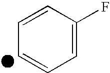 |
| 25 | B | 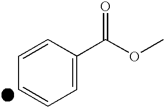 |
| 26 | B | 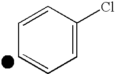 |
| 27 | A | 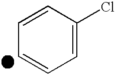 |
| 28 | A | 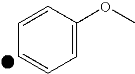 |
| 29 | A | 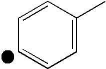 |
| 30 | A | 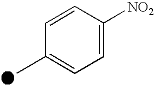 |
| 31 | B | 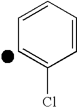 |
| 32 | B | 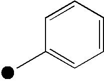 |
| 33 | B | 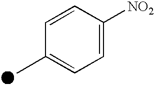 |
| 34 | B | 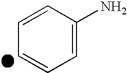 |
| 35 | A | 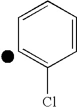 |
| 36 | A | 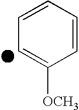 |
| 37 (iii) | B | 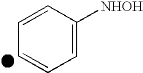 |
| 38 (iii) | C | 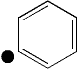 |
| 39 (iii) | C | 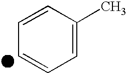 |
| 40 | C | 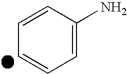 |
| 41 | C | 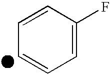 |
| 42 | C | 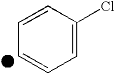 |
| 43 (ii) | C | 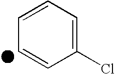 |
| 44 (ii) | A | 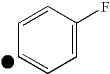 |
| 45 (ii) | A | 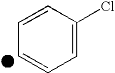 |
| 46 (ii) | A | 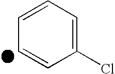 |
| 47 (ii) | C | 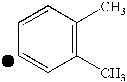 |
| 48 (ii) | C | 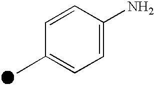 |
| 49 | B | 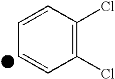 |
| 50 | B | 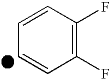 |
| 51 | C | 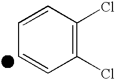 |
| 52 | C | 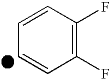 |
| 53 | A | 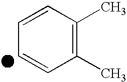 |
| 54 | A | 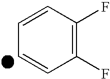 |
| 55 | A | 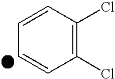 |
| 56 | C | 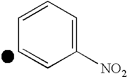 |
| 57 | C | 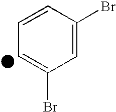 |

R⁴ = A: CH₂OH; B: C(O)NEthyl; C: C(O)NCyclopropyl.
Compounds are of formula (i), unless indicated.

Additional specific values include compounds having the formula (Ib)-(Id) or a pharmaceutically acceptable salt thereof:

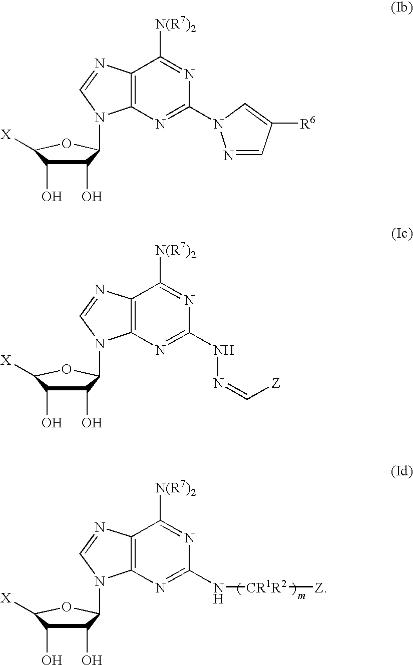

Additional examples of A_{2A} adenosine receptor agonists that are expected to be useful in the present invention include compounds of formula 4:

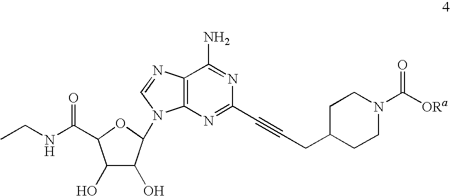

wherein R^{a} is methyl, ethyl, propyl, isopropyl, isobutyl, or t-butyl. Additional examples of A_{2A} adenosine receptor agonists that are expected to be useful in the present invention include those described in U.S. Pat. No. 6,232,297 and in U.S. Patent Application No. 2003/0186926 A1.

Further examples of compounds expected to be useful in the present invention include formula (IA)

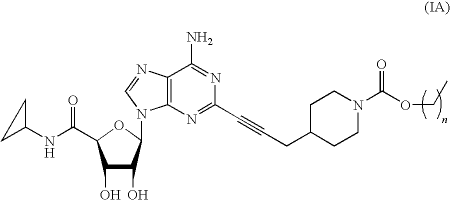

In formula (IA) n is 0, 1, 2, 3, 4, 5, 6, 7, 8, 9, 10, 11, 12, 13, 14, 15, 16, 17, or 18. In another group of specific compounds n is, 5, 6, 7, 8, 9, 10, 11, 12, 13, 14, 15, 16, 17, or 18.

Additional examples of A_{2A} adenosine receptor agonists that are expected to be useful in the present invention include compounds of the invention include formula (IB)

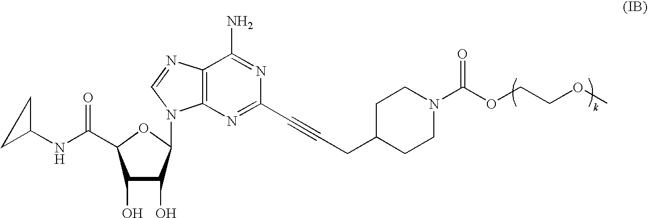

In formula (IB) k is 1, 2, 3, 4, 5, 6, 7, 8, 9, 10, 11, 12, 13, 14, 15, 16, 17, or 18.

Additional examples of A_{2A} adenosine receptor agonists that are expected to be useful in the present invention include compounds of the invention include formula (IC)

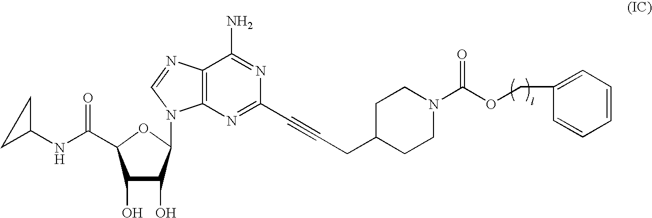

wherein l is 0, 1, 2, 3, or 4.

Other specific compounds of the invention include

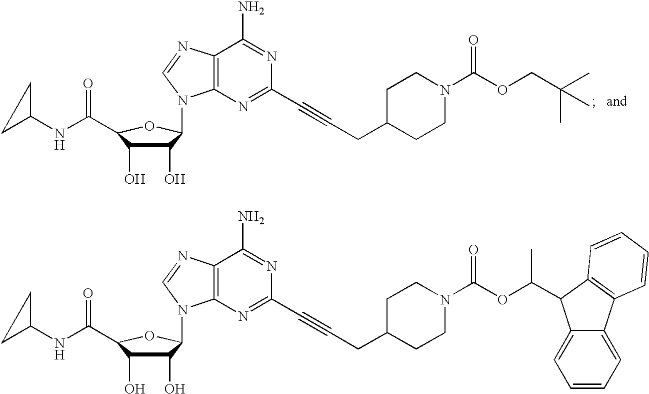

Additional examples of compounds expected to useful in the present invention are illustrated in tables 1, 2, and 3 below:

TABLE 1

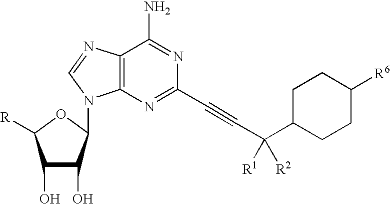

| Compound | R | R¹ | R² | R⁶ |
|---|---|---|---|---|
| ATL2037 | NECA | H | H | CH₂OH |
| MP9056 | NECA | OH | H | CH₂OH |
| ATL146a | NECA | H | H | CO₂H |
| MP9057 | NECA | OH | H | CO₂H |
| ATL146e | NECA | H | H | CO₂Me |
| MP9058 | NECA | OH | H | CO₂Me |
| JR2145 | CH₂OH | H | H | CO₂Me |
| MP9059 | CH₂OH | OH | H | CO₂Me |
| ATL193 | NECA | H | H | CH₂OAc |
| MP9060 | NECA | OH | H | CH₂OAc |
| JR2147 | CH₂OH | H | H | CH₂OAc |
| MP9061 | CH₂OH | OH | H | CH₂OAc |
| JR3023 | NECA | H | H | CH₂N(CH₃)₂ |
| MP9062 | NECA | OH | H | CH₂N(CH₃)₂ |
| JR3021 | NECA | H | H | COOCH₂CH₂NHBoc |
| MP9063 | NECA | OH | H | COOCH₂CH₂NHBoc |
| JR3033 | NECA | H | H | COOCH₂CH₂NH₂ |
| MP9064 | NECA | OH | H | COOCH₂CH₂NH₂ |
| JR3037 | NECA | H | H | CONHCH₂CH₃ |
| MP9065 | NECA | OH | H | CONHCH₂CH₃ |
| JR3055 | NECA | H | H | CONH₂ |
| MP9072 | NECA | OH | H | CONH₂ |
| JR3065 | NECA | H | H | CONHMe |
| MP9066 | NECA | OH | H | CONHMe |
| JR3067B | NECA | H | H | Me, cis CO₂Me |
| MP9067 | NECA | OH | H | Me, cis CO₂Me |
| JR3067A | NECA | H | H | Me, trans CO₂Me |
| MP9068 | NECA | OH | H | Me, trans CO₂Me |
| JR3087 | NECA | H | H | CH₂CH₃ |
| MP9069 | NECA | OH | H | CH₂CH₃ |
| JR3159A | NECA | OH | H | H |
| JR3159B | NECA | OH | H | H |
| JR3119 | NECA | H | H | COCH₃ |
| MP9070 | NECA | OH | H | COCH₃ |
| JR3121 | NECA | H | H | CHCH₃(OH) |
| MP9071 | NECA | OH | H | CHCH₃(OH) |
| JR3139 | NECA | OH | C₆H₁₁ | H |

NECA = CH₃CH₂N(H)C(O)—

TABLE 2

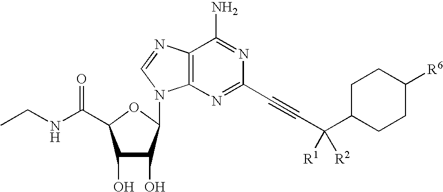

| Compound | R¹ | R² | R⁶ |
|---|---|---|---|
| JR3261 | H | H | H |
| JR3259 | H | H | CO₂tBu |
| JR3269 | H | H | CO₂Et |
| JR4011 | H | H | CO₂iBu |
| JR4009 | H | H | CO₂iPr |
| JR4007 | H | H | COMe |
| JR4051 | H | H | COC(CH₃)₃ |
| JR4047 | H | H | COCH₂(CH₃)₃ |
| MP9047 | H | H | COCH₃ |
| MP9048 | H | H | C(O)N(CH₃)₂ |
| MP9049 | H | H | C(O)N(CH₃)Et |
| MP9050 | H | H | C(O)N(CH₃)iPr |
| MP9051 | H | H | C(O)N(CH₃)iBu |
| MP9052 | H | H | C(O)NH(CH₃) |
| MP9053 | H | H | C(O)NH(Et) |
| MP9054 | H | H | C(O)NH(iPr) |
| MP9055 | H | H | C(O)NH(iBu) |
| TX3261 | OH | H | H |
| TX3259 | OH | H | CO₂tBu |
| TX3269 | OH | H | CO₂Et |
| TX4011 | OH | H | CO₂iBu |
| TX4009 | OH | H | CO₂iPr |
| TX4007 | OH | H | COMe |
| TX4051 | OH | H | COC(CH₃)₃ |
| TX4047 | OH | H | COCH₂(CH₃)₃ |
| TX9047 | OH | H | COCH₃ |
| TX9048 | OH | H | C(O)N(CH₃)₂ |
| TX9049 | OH | H | C(O)N(CH₃)Et |
| TX9050 | OH | H | C(O)N(CH₃)iPr |
| TX9051 | OH | H | C(O)N(CH₃)iBu |
| TX9052 | OH | H | C(O)NH(CH₃) |
| TX9053 | OH | H | C(O)NH(Et) |
| TX9054 | OH | H | C(O)NH(iPr) |
| TX9055 | OH | H | C(O)NH(iBu) |

TABLE 3

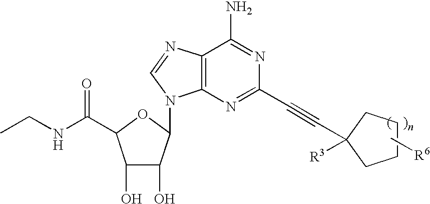

| Compound | n | R³ | R⁶ |
|---|---|---|---|
| JR3135 | 1 | OH | H |
| JR3089 | 2 | OH | H |
| JR3205 | 2 | NH₂ | H |
| JR3177A | 2 | OH | 2-CH₃ |
| JR3177B | 2 | OH | 2-CH₃ |
| JR3181A | 2 | OH | 2-CH₃ |
| JR3181B | 2 | OH | 2-CH₃ |
| JR3227 | 2 | OH | 2-C(CH₃)₃ |
| JR9876 | 2 | OH | 2-C₆H₅ |
| JR3179 | 2 | OH | 3-CH₃ |
| JR3221 | 2 | OH (R) | 3-CH₃ (R) |
| ATL 203 | 2 | OH (S) | 3-CH₃ (R) |
| MP9041 | 2 | OH (R) | 3-CH₃ (S) |
| MP9042 | 2 | OH (S) | 3-CH₃ (S) |
| JR3201B | 2 | OH | 3-(CH₃)₂ |
| MP9043 | 2 | OH (R) | 3-CH₂CH₃ (R) |
| MP9044 | 2 | OH (S) | 3-CH₂CH₃ (R) |
| MP9045 | 2 | OH (R) | 3-CH₂CH₃ (S) |
| MP9046 | 2 | OH (S) | 3-CH₂CH₃ (S) |
| JR3163 | 2 | OH | 3-(CH₃)₂, 5-(CH₃)₂ |
| JR9875 | 2 | OH | 4-CH₃ |
| JR3149 | 2 | OH | 4-C₂H₅ |
| JR3203 | 2 | OH | 4-C(CH₃)₃ |
| JR3161 | 2 | OH | 4-C₆H₅ |

Additional examples of A_{2A} adenosine receptor agonists that are expected to be useful in the present invention include compounds of formula (II):

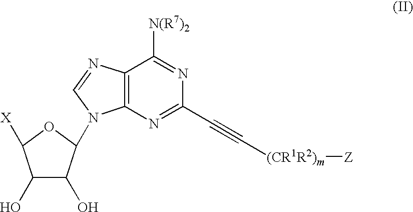

wherein Z is CR³R⁴R⁵; each R¹, R² and R³ is hydrogen; R⁴ and R⁵ together with the carbon atom to which they are attached form a cycloalkyl ring having 3, 4, 5, 6, 7, 8, 9 or 10 ring atoms; and

wherein the ring comprising R⁴ and R⁵ is substituted with —(CH₂)₀₋₆—Y; where Y is —CH₂OR^{a}, —CO₂R^{a}, —OC(O)R^{a}, —CH₂OC(O)R^{a}, —C(O)NR^{b}R^{c}, —CH₂SR^{a}, —C(S)OR^{a}, —OC(S)R^{a}, —CH₂OC(S)R^{a} or C(S)NR^{c} or —CH₂N(R^{b})(R^{c});

each R⁷ is independently hydrogen, (C₁-C₈)alkyl, (C₃-C₈)cycloalkyl, aryl or aryl(C₁-C₈)alkylene;

X is —CH₂OR^{a}, —CO₂R^{a}, —CH₂OC(O)R^{a}, —C(O)NR^{b}R^{c}, —CH₂SR^{a}, —C(S)OR^{a}, —CH₂OC(S)R^{a}, C(S)NR^{b}R^{c} or —CH₂N(R^{b})(R^{c});

each R^{a}, R^{b} and R^{c} is independently hydrogen, (C₁-C₈)alkyl, or (C₁-C₈)alkyl substituted with 1-3 (C₁-C₈)alkoxy, (C₃-C₈)cycloalkyl, (C₁-C₈)alkylthio, amino acid, aryl, aryl(C₁-C₈)alkylene, heteroaryl, or heteroaryl(C₁-C₈)alkylene; or R^{b} and R^{c}, together with the nitrogen to which they are attached, form a pyrrolidino, piperidino, morpholino, or thiomorpholino ring; and m is 0 to about 6; or a pharmaceutically acceptable salt thereof.

A specific value for —N(R⁷)₂ is amino, monomethylamino or cyclopropylamino.

A specific value for Z is carboxy- or —(C₁-C₄)alkoxycarbonyl-cyclohexyl(C₁-C₄)alkyl.

A specific value for R^{a} is H or (C₁-C₄)alkyl, i.e., methyl or ethyl.

A specific value for R^{b} is H, methyl or phenyl.

A specific value for R^{c} is H, methyl or phenyl.

A specific value for —(CR¹R²)ₘ— is —CH₂— or —CH₂—CH₂—.

A specific value for X is CO₂R^{a}, (C₂-C₅)alkanoylmethyl or amido.

A specific value for Y is CO₂R^{a}, (C₂-C₅)alkanoylmethyl or amido.

A specific value for m is 1.

Specific compounds expected to be useful for practicing the invention are compounds JR3259, JR3269, JR4011, JR4009, JR-1085 and JR4007.

Specific A_{2A} adenosine receptor agonists expected to be useful in the present invention having formula (II) include those described in U.S. Pat. No. 6,232,297.

Specific compounds of formula (II) are those wherein each R⁷ is H, X is ethylaminocarbonyl and Z is 4-carboxycyclohexylmethyl (DWH-146a), Z is 4-methoxycarbonylcyclohexylmethyl (DWH-146e), Z is 4-isopropylcarbonylcyclohexylmethyl (AB-1), Z is 4-acetoxymethyl-cyclohexylmethyl (JMR-193) or Z is 4-pyrrolidine-1-carbonylcyclohexylmethyl (AB-3).

Additional examples of A_{2A} adenosine receptor agonists that are expected to be useful in the present invention include those depicted below.

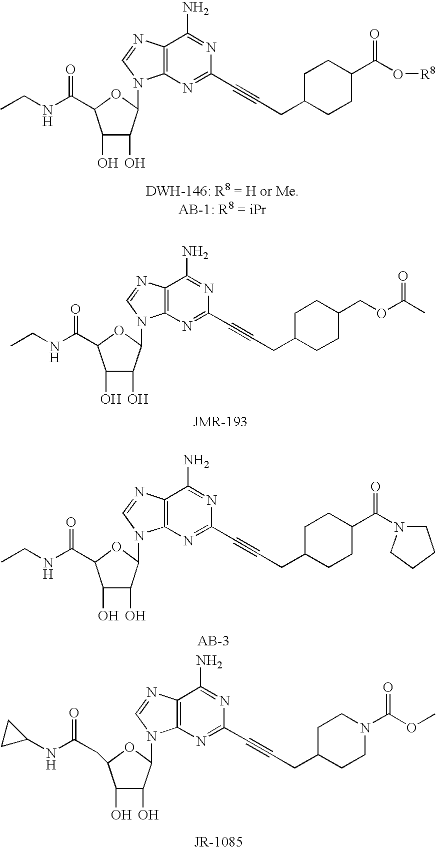

Additional examples of A_{2A} adenosine receptor agonists of formula (II) that are expected to be useful in the present invention include those described in U.S. Pat. No. 6,232,297. These compounds, having formula (II), can be prepared according to the methods described therein.

Another specific group of agonists of A_{2A} adenosine receptors that are expected to be useful in the practice of the present invention include compounds having the general formula (III):

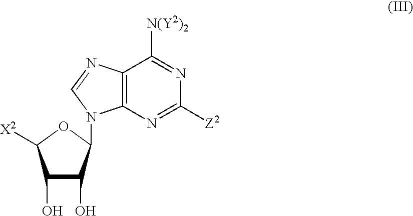

wherein Z² is a group selected from the group consisting of —OR¹², —NR¹³R¹⁴, a —C/C—Z³, and —NH—N═R¹⁷;

each Y² is individually H, C₁-C₆ alkyl, C₃-C₇ cycloalkyl, phenyl or phenyl C₁-C₃ alkyl;

R¹² is C₁₋₄-alkyl; C₁₋₄-alkyl substituted with one or more C₁₋₄-alkoxy groups, halogens (fluorine, chlorine or bromine), hydroxy groups, amino groups, mono(C₁₋₄-alkyl)amino groups, di(C₁₋₄-alkyl)amino groups or C₆₋₁₀-aryl groups wherein the aryl groups may be substituted with one or more halogens (fluorine, chlorine or bromine), C₁₋₄-alkyl groups, hydroxy groups, amino groups, mono(C₁₋₄-alkyl)amino groups or di(C₁₋₄-alkyl)amino groups); or C₆₋₁₀-aryl; or C₆₋₁₀-aryl substituted with one or more halogens (fluorine, chlorine or bromine), hydroxy groups, amino groups, mono(C₁₋₄-alkyl)amino groups, di(C₁₋₄-alkyl)amino groups or C₁₋₄-alkyl groups;

one of R¹³ and R¹⁴ has the same meaning as R¹² and the other is hydrogen; and

R¹⁷ is a group having the formula (I)

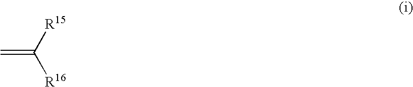

wherein each of R¹⁵ and R¹⁶ independently may be hydrogen, (C₃-C₇)cycloalkyl or any of the meanings of R¹², provided that R¹⁵ and R¹⁶ are not both hydrogen;

X² is CH₂OH, CH₃, CO₂R²⁰ or C(═O)NR²¹R²² wherein R²⁰ has the same meaning as R¹³ and wherein R²¹ and R²² have the same meanings as R¹⁵ and R¹⁶ or R²¹ and R²² are both H;

Z³ has one of the following meanings:

C₆-C₁₀ aryl, optionally substituted with one to three halogen atoms, C₁-C₆ alkyl, C₁-C₆ haloalkyl, C₁-C₆ alkoxy, C₁-C₆ haloalkoxy, C₂-C₆ alkoxycarbonyl, C₂-C₆ alkoxyalkyl, C₁-C₆ alkylthio, thio, CHO, cyanomethyl, nitro, cyano, hydroxy, carboxy, C₂-C₆ acyl, amino C₁-C₃ monoalkylamino, C₂-C₆ dialkylamino, methylenedioxy or aminocarbonyl;

a group of formula —(CH₂)_{q}-Het wherein q is 0 or an integer from 1 to 3 and Het is 5 or 6 membered heterocyclic aromatic or non-aromatic ring, optionally benzocondensed, containing 1 to 3 heteroatoms selected from non-peroxide oxygen, nitrogen or sulphur, linked through a carbon atom or through a nitrogen atom;

C₃-C₇ cycloalkyl optionally containing unsaturation or C₂-C₄ alkenyl;

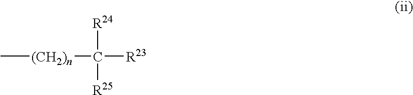

wherein

R²³ is hydrogen, methyl or phenyl;

R²⁴ is hydrogen, C₁-C₆ linear or branched alkyl, C₅-C₆ cycloalkyl or C₃-C₇ cycloalkenyl, phenyl-C₁-C₂-alkyl or R²³ and R²⁴, taken together, form a 5 or 6-membered carbocyclic ring or R²⁵ is hydrogen and R²³ and R²⁴, taken together, form an oxo group or a corresponding acetalic derivative;

R²⁵ is OH, NH₂ dialkylamino, halogen, cyano; and n is 0 or 1 to 4; or C₁-C₁₆ alkyl, optionally comprising 1-2 double bonds, O, S or NY²;

or a pharmaceutically acceptable salt thereof.

Specific C₆₋₁₀-aryl groups include phenyl and naphthyl.

Additional specific values include compounds wherein in the compound of formula (III), Z² is a group of the formula (iii)

—O—(CH₂)ₙ—Ar (iii)

wherein n is an integer from 1-4, e.g., 2, and Ar is a phenyl group, tolyl group, naphthyl group, xylyl group or mesityl group. In one embodiment, Ar is a para-tolyl group and n=2.

Additional specific values include compounds wherein in the compound of formula (III), Z² is a group of the formula (Iv)

NHN═CHCy (iv)

wherein Cy is a C₃₋₇-cycloalkyl group, such as cyclohexyl or a C₁₋₄ alkyl group, such as isopropyl.

Additional specific values include compounds wherein in the compound of formula (III), Z² is a group of the formula (vii)

C≡CZ³ (v)

wherein Z³ is C₃-C₁₆ alkyl, hydroxy C₂-C₆ alkyl or (phenyl) (hydroxymethyl).

Additional examples of compounds of formula (III) include those shown below:

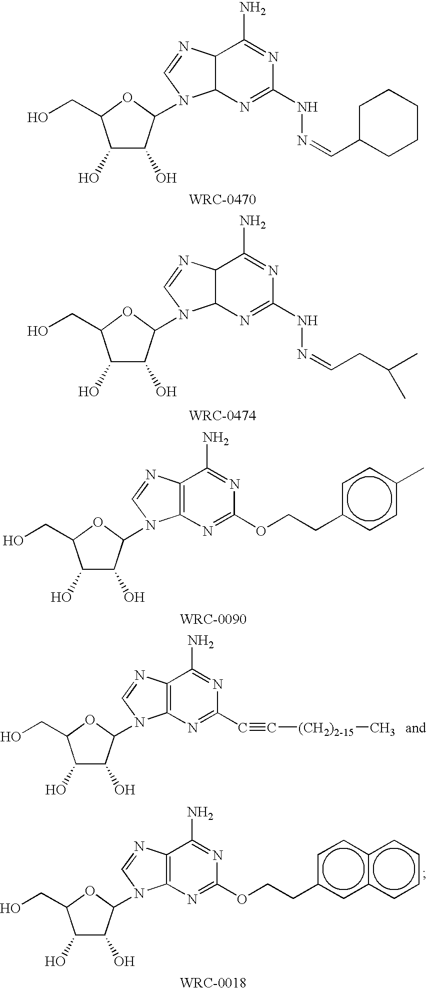

wherein the H on CH₂OH can optionally be replaced by ethylaminocarbonyl. Of these specific examples, WRC-0474[SHA 211] and WRC-0470 are particularly preferred.

Such compounds may be synthesized as described in: Olsson et al. (U.S. Pat. Nos. 5,140,015 and 5,278,150); Cristalli (U.S. Pat. No. 5,593,975); Miyasaka et al. (U.S. Pat. No. 4,956,345); Hutchinson, A. J. et al., J. Pharmacol. Exp. Ther., 251, 47 (1989); Olsson, R. A. et al., J. Med. Chem., 29, 1683 (1986); Bridges, A. J. et al., J. Med. Chem., 31, 1282 (1988); Hutchinson, A. J. et al., J. Med. Chem., 33, 1919 (1990); Ukeeda, M. et al., J. Med. Chem., 34, 1334 (1991); Francis, J. E. et al., J. Med. Chem., 34, 2570 (1991); Yoneyama, F. et al., Eur. J. Pharmacol., 213, 199-204 (1992); Peet, N. P. et al., J. Med. Chem., 35, 3263 (1992); and Cristalli, G. et al., J. Med. Chem., 35, 2363 (1992); all of which are incorporated herein by reference.

Additional specific values include compounds having formula (III) where Z² is a group having formula (vi):

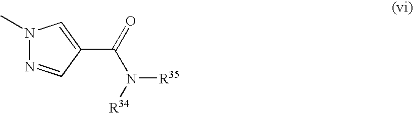

wherein R³⁴ and R³⁵ are independently H, C₁-C₆ alkyl, C₃-C₇ cycloalkyl, phenyl, phenyl C₁-C₃ alkyl or R³⁴ and R³⁵ taken together with the nitrogen atom are a 5- or 6-membered heterocyclic ring containing 1-2 heteroatoms selected from non-peroxide oxygen, nitrogen (N(R¹³)) or sulphur atoms. In one embodiment, one of R³⁴ and R³⁵ is hydrogen and the other is ethyl, methyl or propyl. In another embodiment, one of R³⁴ and R³⁵ is hydrogen and the other is ethyl or methyl.

A specific pyrazole derivative that is expected to be useful in practicing the present invention is a compound having the formula:

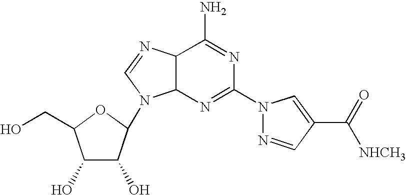

Another specific group of agonists of A_{2A} adenosine receptors that are expected to be useful in the present invention include compounds having the general formula (IV):

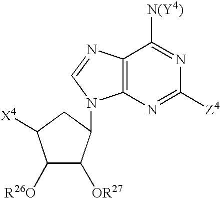

wherein Z⁴ is —NR²⁸R²⁹;

R²⁸ is hydrogen or (C₁-C₄)alkyl; and R²⁹ is
a) (C₁-C₄)alkyl;
b) (C₁-C₄)alkyl substituted with one or more (C₁-C₄)alkoxy, halogen, hydroxy, amino, mono((C₁-C₄)alkyl)amino, di((C₁-C₄)alkyl)amino or (C₆-C₁₀)aryl wherein aryl is optionally substituted with one or more halogen, hydroxy, amino, (C₁-C₄)alkyl, R³⁰OOC—((C₁-C₄)alkyl)-, R³¹R³²NC(═O)—((C₁-C₄)alkyl)-, mono((C₁-C₄)alkyl)amino or di((C₁-C₄)alkyl)amino;
c) (C₆-C₁₀)aryl; or
d) (C₆-C₁₀)aryl substituted with one or more halogen, hydroxy, amino, mono((C₁-C₄)alkyl)amino, di((C₁-C₄) alkyl)amino or (C₁-C₄)alkyl;

wherein each Y⁴ is individually H, (C₁-C₆)alkyl, (C₃-C₇)cycloalkyl, phenyl or phenyl(C₁-C₃)alkyl; and X⁴ is —C(═O)NR³¹R³², —COOR³⁰, or —CH₂OR³⁰;

wherein each of R³¹ and R³² are independently; hydrogen; C₃₋₇-cycloalkyl; (C₁-C₄)alkyl; (C₁-C₄)alkyl substituted with one or more (C₁-C₄)alkoxy, halogen, hydroxy, —COOR³³, amino, mono((C₁-C₄)alkyl)amino, di((C₁-C₄)alkyl)amino or (C₆-C₁₀)aryl wherein aryl is optionally substituted with one or more halogen, (C₁-C₄)alkyl, hydroxy, amino, mono((C₁-C₄) alkyl)amino or di((C₁-C₄) alkyl)amino; (C₆-C₁₀)aryl; or (C₆-C₁₀)aryl substituted with one or more halogen, hydroxy, amino, mono((C₁-C₄)alkyl)amino, di((C₁-C₄)alkyl)amino or (C₁-C₄)alkyl;

R²⁶ and R²⁷ independently represent hydrogen, lower alkanoyl, lower alkoxy-lower alkanoyl, aroyl, carbamoyl or mono- or di-lower alkylcarbamoyl; and R³⁰ and R³³ are independently hydrogen, (C₁-C₄)alkyl, (C₆-C₁₀)aryl or (C₆-C₁₀)aryl((C₁-C₄)alkyl); or a pharmaceutically acceptable salt thereof.

Additional specific values include compounds wherein at least one of R²³ and R²⁹ is (C₁-C₄)alkyl substituted with one or more (C₁-C₄)alkoxy, halogen, hydroxy, amino, mono((C₁-C₄)alkyl)amino, di((C₁-C₄)alkyl)amino or (C₆-C₁₀)aryl wherein aryl is optionally substituted with one or more halogen, hydroxy, amino, (C₁-C₄)alkyl, R³⁰OOC—(C₁-C₄)alkyl, mono((C₁-C₄)alkyl)amino or di((C₁-C₄)alkyl)amino.

Additional specific values include compounds wherein at least one of R³¹ and R³² is C₁₋₄-alkyl substituted with one or more (C₁-C₄)alkoxy, halogen, hydroxy, amino, mono((C₁-C₄)alkyl)amino, di((C₁-C₄)alkyl)amino or C₆₋₁₀-aryl wherein aryl is optionally substituted with one or more halogen, hydroxy, amino, (C₁-C₄)alkyl, R³⁰C—(C₁-C₄)alkylene-, mono((C₁-C₄)alkyl)amino or di((C₁-C₄)alkyl)amino. Additional specific values include compounds wherein at least one of R²⁸ and R²⁹ is C₆₋₁₀-aryl substituted with one or more halogen, hydroxy, amino, mono((C₁-C₄)alkyl)amino, di((C₁-C₄) alkyl)amino or (C₁-C₄)alkyl.

Additional specific values include compounds wherein at least one of R³¹ and R³² is C₆₋₁₀-aryl substituted with one or more halogen, hydroxy, amino, mono((C₁-C₄)alkyl)-amino, di((C₁-C₄)alkyl)amino or (C₁-C₄)alkyl.

Additional specific values include compounds wherein R³¹ is hydrogen and R³² is (C₁-C₄)alkyl, cyclopropyl or hydroxy-(C₂-C₄)alkyl. A specific R²⁸ group is (C₁-C₄)alkyl substituted with (C₆-C₁₀)aryl, that is in turn substituted with R³⁰O(O)C—(C₁-C₄)alkylene-.

A specific compound having formula (IV) is:

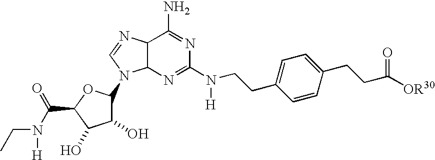

wherein R³⁰ is hydrogen, methyl, ethyl, n-propyl or isopropyl. One embodiment provides a compound wherein the R³⁰ group is methyl or ethyl. In one embodiment, the R³⁰ group is methyl.

Two compounds that can be used in practicing the present invention have the formula:

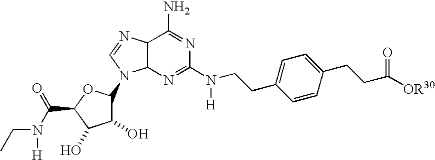

wherein R³⁰ is hydrogen (acid, CGS21680) and where R³⁰ is methyl (ester, JR2171).

The compounds of the invention having formula (IV) may be synthesized as described in: U.S. Pat. No. 4,968,697 or J. Med. Chem., 33, 1919-1924, (1990).

Another agonist compound expected to be useful in the present invention is IB-MECA:

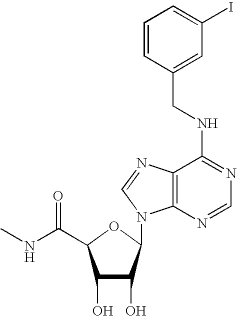

The compounds of formulas described herein, e.g., (I), (II), (III), and (IV), may have more than one chiral center and may be isolated in optically active and racemic forms. In one embodiment, the riboside moiety of the compounds is derived from D-ribose, i.e., the 3N,4N-hydroxyl groups are alpha to the sugar ring and the 2N and 5N groups is beta (3R, 4S, 2R, 5S). When the two groups on the cyclohexyl group are in the 1- and 4-position, they are preferably trans. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, or enzymatic techniques, by synthesis from optically-active starting materials, by chiral synthesis, or by chromatographic separation using a chiral stationary phase) and how to determine adenosine agonist activity using the tests described herein, or using other similar tests which are well known in the art.

### DEFINITIONS

The following definitions are used, unless otherwise described.

Mammal or subject includes human, equine, porcine, canine, and feline.

A_{2A} agonist refers to an agent that activates the Adenosine A_{2A} receptor with a Ki of <1 μM. An A_{2A} agonist may be selective for A_{2A} (e.g., at least 10, 50, or 100/1 over another adenosine receptor subtype/A₂A receptor). An A_{2A} agonist may also be cross reactive with other adenosine receptor subtypes (e.g., A₁, A_{2B}, and A₃). The A_{2A} agonist may activate other receptors with a greater or lesser affinity than the A_{2A} receptor.

By “pathological pain” is meant any pain resulting from pathology, such as from functional disturbances and/or pathological changes, injuries, lesions, burns and the like. One form of pathological pain is “neuropathic pain.” The term “neuropathic pain” refers to pain caused by, but not limited to, a neuropathy, an encephalopathy and/or a myelopathy (i.e., functional disturbances or pathological states of the peripheral nervous system, brain and spinal cord, respectively). Neuropathic pain can be caused by nerve damage, injury such as spinal cord injury, neuritis, inflammation, noninflammatory lesions, electrical injuries, headaches, and the like. Neuropathic pain can also be caused by complications of various diseases, including without limitation, demyelinating diseases, diabetes, amyloid diseases, porphyric diseases, Lyme disease, leprosy, acromegaly, rheumatoid arthritis, autoimmune diseases, metabolic diseases, cancer, and viral infection. Such pain can also be caused by toxic states, such as but not limited to, toxic states caused by arsenic, isoniazid, lead and nitrofurantoin. Examples of neuropathic pain include, but are not limited to, thermal or mechanical hyperalgesia, thermal or mechanical allodynia, diabetic pain, pain arising from irritable bowel or other internal organ disorders, endometriosis pain, phantom limb pain, complex regional pain syndromes, fibromyalgia, low back pain, cancer pain, pain arising from infection, inflammation or trauma to peripheral nerves or the central nervous system, multiple sclerosis pain, entrapment pain, pain from HIV infection, herpesvirus infection, and the like.

“Hyperalgesia” means an abnormally increased pain sense, such as pain that results from an excessive sensitiveness or sensitivity.

“Hypalgesia” (or “hypoalgesia”) means the decreased pain sense.

“Allodynia” means pain that results from a non-noxious stimulus to the skin. Examples of allodynia include, but are not limited to, cold allodynia, tactile allodynia, and the like.

“Nociception” is defined herein as pain sense.

“Nociceptor” herein refers to a structure that mediates nociception. The nociception may be the result of a physical stimulus, such as, mechanical, electrical, thermal, or a chemical stimulus. Nociceptors are present in virtually all tissues of the body.

“Analgesia” is defined herein as the relief of pain without the loss of consciousness. An “analgesic” is an agent or drug useful for relieving pain, again, without the loss of consciousness.

Halo is fluoro, chloro, bromo, or iodo.

Alkyl, alkoxy, aralkyl, alkylaryl, etc. denote both straight and branched alkyl groups; but reference to an individual radical such as “propyl” embraces only the straight chain radical, a branched chain isomer such as “isopropyl” being specifically referred to.

Aryl denotes a phenyl radical or an ortho-fused bicyclic carbocyclic radical having about nine to ten ring atoms in which at least one ring is aromatic. Heteroaryl denotes a radical of a monocyclic aromatic ring containing five or six ring atoms consisting of carbon and 1, 2, 3, or 4 heteroatoms each selected from the group consisting of non-peroxide oxygen, sulfur, and N(Y) wherein Y is absent or is H, O, (C₁-C₈)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

Heteroaryl encompasses a monocyclic aromatic ring having five or six ring atoms consisting of carbon and 1-4 heteroatoms each selected from the group consisting of non-peroxide oxygen, sulfur, and N(X) wherein X is absent, is H, O, (C₁-C₄)alkyl, phenyl or benzyl, or is a substituent defined elsewhere. Heteroaryl also encompasses a radical of an ortho-fused bicyclic heterocycle of 8-10 ring atoms, particularly a benz-derivative or one derived by fusing a propylene, trimethylene, or tetramethylene diradical thereto. Only one ring of the bicyclic heteroaryl need be aromatic.

The term “heterocycle” generally represents a non aromatic heterocyclic group, having from 3 to about 10 ring atoms, which can be saturated or partially unsaturated, containing at least one heteroatom (e.g., 1, 2, or 3) selected from the group consisting of oxygen, nitrogen, and sulfur. Specific, “heterocycle” groups include monocyclic, bicyclic, or tricyclic groups containing one or more heteroatoms selected from the group consisting of oxygen, nitrogen, and sulfur. A “heterocycle” group also can include one or more oxo groups (═O) attached to a ring atom. Non-limiting examples of heterocycle groups include 1,3-dioxolane, 1,4-dioxane, 1,4-dithiane, 2H-pyran, 2-pyrazoline, 4H-pyran, chromanyl, imidazolidinyl, imidazolinyl, indolinyl, isochromanyl, isoindolinyl, morpholine, piperazinyl, piperidine, piperidyl, pyrazolidine, pyrazolidinyl, pyrazolinyl, pyrrolidine, pyrroline, quinuelidine, thiomorpholine, and the like.

The term carbocyclic biaryl refers to ortho-fused bicyclic moieties, typically containing 10 carbon atoms. An example is naphthalene. The term heterocyclic biaryl as used herein refers to ortho-fused bicyclic moieties containing 1-4 heteroatoms. Examples include indoles, isoindoles, quinolines, isoquinolines, benzofurans, isobenzofurans, benzothiophenes, benzo[c]thiophenes, benzimidazoles, purines, indazoles, benzoxazole, benzisoxazole, benzothiazole, quinoxalines, quinazolines, cinnolines, and the like.

The point of attachment of either the carbocyclic or heterocyclic biaryl can be to any ring atom permitted by the valency of that atom.

Specific and preferred values listed below for radicals, substituents, and ranges, are for illustration only; they do not exclude other defined values or other values within defined ranges for the radicals and substituents.

Carbon chains and their optionally substituted counterparts can be in any branched chain form permitted by the valencies and steric requirements of the atoms. Specifically, (C₁-C₈)alkyl can be methyl, ethyl, propyl, isopropyl, butyl, iso-butyl, sec-butyl, tert-butyl, pentyl, 3-pentyl, neopentyl, hexyl, heptyl, octyl, and the like, in any branched chain form.

As used herein, the term “cycloalkyl” encompasses bicycloalkyl (norbornyl, 2,2,2-bicyclooctyl, etc.) and tricycloalkyl (adamantyl, etc.), optionally comprising 1-2 N, O or S. Cycloalkyl also encompasses (cycloalkyl)alkyl. Thus, (C₃-C₆)cycloalkyl can be cyclopropyl, cyclobutyl, cyclopentyl, cyclohexyl and the like. (C₁-C₈)alkoxy can be methoxy, ethoxy, propoxy, isopropoxy, butoxy, iso-butoxy, sec-butoxy, pentoxy, 3-pentoxy, or hexyloxy, in any branched chain form.

(C₂-C₆)alkenyl can be vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl.

(C₁-C₆)alkanoyl can be acetyl, propanoyl or butanoyl; halo(C₁-C₆)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; hydroxy(C₁-C₆)alkyl can be hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 1-hydroxypentyl, 5-hydroxypentyl, 1-hydroxyhexyl, or 6-hydroxyhexyl.

(C₁-C₆)alkoxycarbonyl (CO₂R²) can be methoxycarbonyl, ethoxycarbonyl, propoxycarbonyl, isopropoxycarbonyl, butoxycarbonyl, pentoxycarbonyl, or hexyloxycarbonyl.

(C₁-C₆)alkylthio can be methylthio, ethylthio, propylthio, isopropylthio, butylthio, isobutylthio, pentylthio, or hexylthio.

(C₂-C₆)alkanoyloxy can be acetoxy, propanoyloxy, butanoyloxy, isobutanoyloxy, pentanoyloxy, or hexanoyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyraxolyl, pyrrolyl, pyrazinyl, tetrazolyl, puridyl (or its N-oxide), thienyl, pyrimidinyl (or its N-oxide), indolyl, isoquinolyl (or its N-oxide) or quinolyl (or its N-oxide).

The term “alkylene” refers to a divalent straight or branched hydrocarbon chain (e.g. ethylene —CH₂CH₂—).

The term “aryl(C₁-C₈)alkylene” for example includes benzyl, phenethyl, 3-phenylpropyl, naphthylmethyl and the like.

“Treating” or “treatment” covers the treatment of a disease-state in a mammal, and includes: (a) preventing the disease-state from occurring in a mammal, in particular, when such mammal is predisposed to the disease-state but has not yet been diagnosed as having it; (b) inhibiting the disease-state, e.g., arresting it development; and/or (c) relieving the disease-state, e.g., causing regression of the disease state until a desired endpoint is reached. Treating also includes the amelioration of a symptom of a disease (e.g., lessen the pain or discomfort), wherein such amelioration may or may not be directly affecting the disease (e.g., cause, transmission, expression, etc.).

As used herein the term “in conjunction with” refers to co-administration of an anti-rejection agent with the A_{2A} adenosine receptor agonist. The co-administration of an agent and an A_{2A} adenosine receptor agonists includes administration of the agent and agonist either simultaneously, as a mixture, or sequentially. The sequential administration of the A_{2A} adenosine receptor agonists can be prior to administration of the agent, within minutes or up to about 48 hours either before the administration of the agent. The A_{2A} adenosine receptor agonists can also be administered after the agent. Preferably the administration of the A_{2A} adenosine receptor agonists will be within about 24 hours and more preferably within about 12 hours.

The carbon atom content of various hydrocarbon-containing moieties is indicated by a prefix designating the minimum and maximum number of carbon atoms in the moiety, i.e., the prefix Cᵢ-Cⱼ indicates a moiety of the integer “i” to the integer “j” carbon atoms, inclusive. Thus, for example, (C₁-C₈)alkyl refers to alkyl of one to eight carbon atoms, inclusive.

The compounds of the present invention are generally named according to the IUPAC or CAS nomenclature system. Abbreviations which are well known to one of ordinary skill in the art may be used (e.g., “Ph” for phenyl, “Me” for methyl, “Et” for ethyl, “h” for hour or hours and “rt” for room temperature).

It will be appreciated by those skilled in the art that the compounds described herein may have more than one chiral center and may be isolated in optically active and racemic forms. Preferably, the riboside moiety is derived from D-ribose. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, or enzymatic techniques, by synthesis from optically-active starting materials, by chiral synthesis, or by chromatographic separation using a chiral stationary phase) and how to determine adenosine agonist activity using the tests described herein, or using other similar tests which are well known in the art.

In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Formulation and Dosages

The compounds of the present invention can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

The pharmaceutical compositions also comprising a pharmaceutically acceptable excipient (e.g., carrier).

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain: binders, such as gum tragacanth, acacia, corn starch or gelatin; excipients, such as dicalcium phosphate; a disintegrating agent, such as corn starch, potato starch, alginic acid and the like; a lubricant, such as magnesium stearate; and a sweetening agent, such as sucrose, fructose, lactose or aspartame or a flavoring agent, such as peppermint, oil of wintergreen, or cherry flavoring. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various other ingredients as enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid, a liquid or in a dermatological patch.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds for the present invention can be determined by comparing their in vitro activity, and in vivo activity in animal models. Methods for the extrapolation of effective dosages in mice, and other animals, to humans are known to the art; for example, see U.S. Pat. No. 4,938,949. Useful dosages of Type IV PDE inhibitors are known to the art. For example, see, U.S. Pat. No. 5,877,180, Col. 12.

Generally, the concentration of the compounds for the present invention in a liquid composition, such as a lotion, will be from about 0.1-25% wt-%, preferably from about 0.5-10 wt-%. The concentration in a semi-solid or solid composition such as a gel or a powder will be about 0.1-5 wt-%, preferably about 0.5-2.5 wt-%.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 μg/kg, e.g., from about 10 to about 75 μg/kg of body weight per day, such as 3 to about 50 μg per kilogram body weight of the recipient per day, preferably in the range of 6 to 90 μg/kg/day, most preferably in the range of 15 to 60 μg/kg/day.

The compound is conveniently administered in unit dosage form; for example, containing 5 to 1000 μg, conveniently 10 to 750 μg, most conveniently, 50 to 500 μg of active ingredient per unit dosage form.

Ideally, the active ingredient should be administered to achieve peak plasma concentrations of the active compound of from about 0.1 to about 10 nM, preferably, about 0.2 to 10 nM, most preferably, about 0.5 to about 5 nM. This may be achieved, for example, by the intravenous injection of a 0.05 to 5% solution of the active ingredient, optionally in saline, or orally administered as a bolus containing about 1-100 μg of the active ingredient. Desirable blood levels may be maintained by continuous infusion to provide about 0.01-5.0 μg/kg/hr or by intermittent infusions containing about 0.4-15 μg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye. For example, it is desirable to administer the present compositions intravenously over an extended period of time following the insult that gives rise to inflammation.

The ability of a given compound of the invention to act as an A_{2A} adenosine receptor agonist may be determined using pharmacological models which are well known to the art, or using tests described below.

The invention will be further described by reference to the following detailed examples, which are given for illustration of the invention, and are not intended to be limiting thereof.

### EXAMPLES

A_{2A} adenosine receptor agonists useful in the present invention can be prepared as shown in the patents and publications described herein (e.g., U.S. Pat. No. 4,968,697; U.S. Pat. No. 4,956,345; U.S. Pat. No. 5,140,015; U.S. Pat. No. 5,278,150; U.S. Pat. No. 5,593,975; U.S. Pat. No. 6,232,297; U.S. Pat. No. 6,403,567; U.S. Pat. No. 6,642,210; U.S. Pat. No. 7,214,665; U.S. Pat. Appl. No. 2006/004088; and, U.S. Pat. Appl. No. 2007/0270373). Additional A_{2A} agonists are known in the art and are expected to be useful in the present invention. Furthermore, assays to determine whether or not an agent functions as an A_{2A} agonist are well known in the art (e.g., see the above list of patents and publications).

Pain Methodology:

Saline is used as the vehicle in the experiments. All A_{2A} agonists are dissolved in 100% DMSO to a 10 mM concentration. These are then diluted 1:10,000 with saline. The total volume of injection for all groups is 5 μL, which consists of a 1 μL air bubble, 1 μL of agonist/vehicle, 1 μL air bubble, and finally a 2 μL flush of saline. The intermediate air bubble is used to separate drug/vehicle and the flush.

### Example 1

### Administration of A_{2A} Agonists

Sprague Dawley rats underwent chronic constriction injury (CCI) of the sciatic nerve or sham surgery. After pre-surgery baseline testing (Day 0=D0), rats received chronic constriction injury of the left sciatic nerve at mid-thigh level to produce neuropathic pain (chronic constriction injury model: CCI). This is seen by the fall in pain threshold between days 4 and 11 (D4, D11) after surgery relative to D0. Once CCI-induced allodynia was stable as tested by von Frey filaments, the material to be studied (e.g., vehicle or A_{2A}R agonists CGS21680 or ATL313) was injected intrathecally. After injection, behavioral testing occurred at 4, 24, and 72 h and then weekly for 6 weeks.

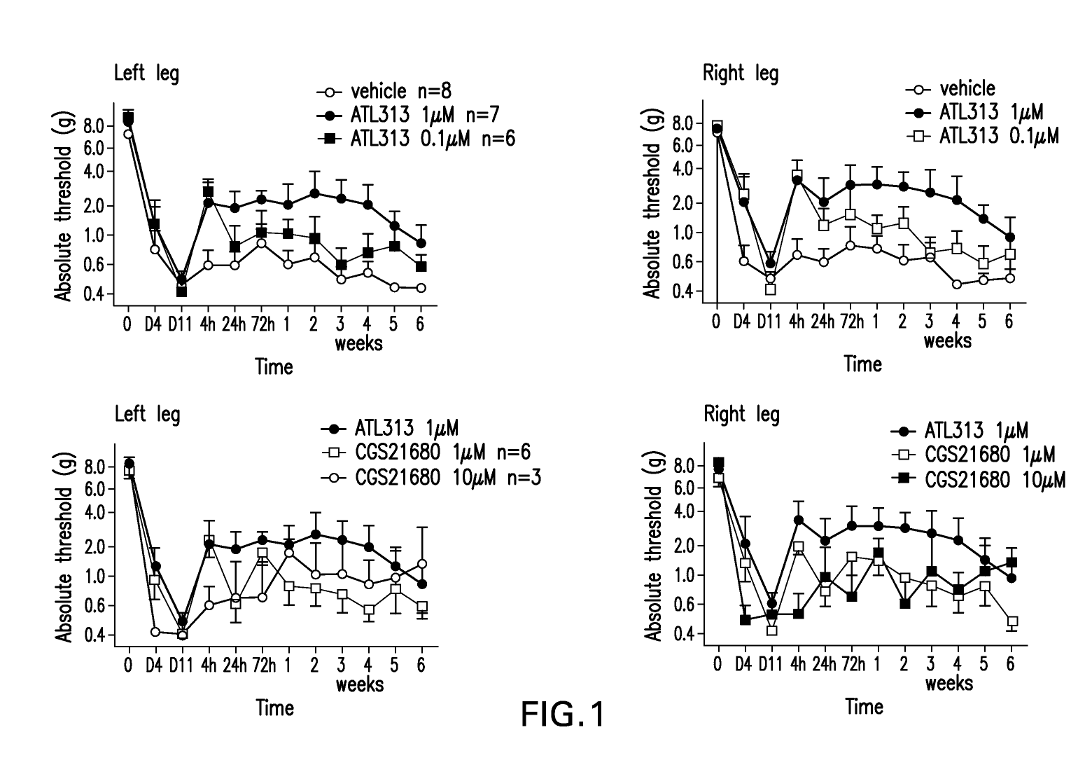

The results of the studies are shown in FIG. 1 with the translation of Y-axis units as follows: 5=10 grams, 4.75=5.62 grams, 4.5=3.16 grams, 4.25=1.73 grams, 4=1 gram, 3.75=0.56 grams, 3.5=0.32 grams.

### Example 2

### Blockade and Reversal of A_{2A} Agonist by an Antagonist (ZM241385)

CCI surgery and indwelling intrathecal catheters were implanted in male Sprague-Dawley rats (325-350 g, n=6/group). 10-14 days after surgery, when the allodynia is stable, an A_{2A} antagonist (ZM241385, 10 uM, Tocris Bioscience) or vehicle was co-administered with ATL313 or vehicle. von Frey testing was done before surgery, before intrathecal injections, and 1, 2, 3, 4, 6, and 24 h after injection.

In a separate group of animals, ATL313 (1 uM) was administered 10-14 days after CCI surgery. One week after ATL313 (1 uM, i.t.) administration, ZM241385 (10 uM) or equivolume vehicle was administered intrathecally. von Frey testing was done 1, 2, 3, 4, 6 & 24 h after injection.

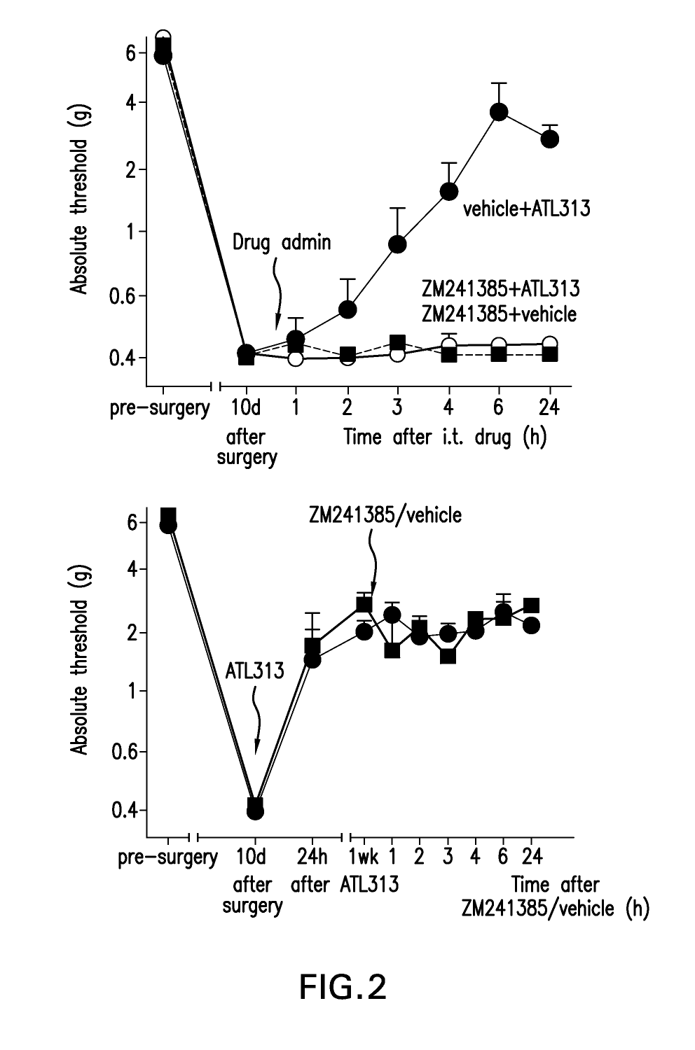

FIG. 2, top panel, demonstrates that co-administration of ATL313 and ZM241385, 10-14 days after CCI surgery, abolishes the effect of ATL313 on the CCI-induced allodynia (P<0.0001). Administration of a ten-fold higher dose of the A_{2A} antagonist (ZM241385, 10 uM), to that of the A_{2A} agonist (1 uM), has no effect on the CCI-induced allodynia (P>0.05). Our results show that the effect of co-administration of ATL313 (1 uM) and an A_{2A} antagonist (ZM241385, 10 uM) completely abolishes the effect of the A_{2A} agonist alone. Therefore, the effect of ATL313 on neuropathic allodynia is indeed believed to be A_{2A} receptor mediated.

FIG. 2, bottom panel, demonstrates that the A_{2A} antagonist ZM241385 had no effect on reversal of the allodynia induced by the previous ATL313 administration when administered one week later. Our results infer that the initial reversal of neuropathic allodynia is triggered by A_{2A} receptor agonism, but that the long-lasting effects, when the drug is no longer present, is possibly from long-lasting intracellular changes, triggered by the initial A_{2A} receptor activity.

### Example 3

### Dose Response of ATL313 and Comparison with Other A_{2A} Agonists

The mechanical sensitivity to von Frey filaments applied to the plantar surface of the hind paw, measured in grams, in animals following unilateral CCI surgery of the left sciatic nerve increases significantly by 10 days, and remains stable for at least 9 wks following surgery (not shown). A single intrathecal injection of ATL313 (1 uM) given 10-14 days after CCI surgery when the allodynia is stable, results in a partial reversal of the allodynia for at least 4 weeks (P<0.05). ATL313, is not analgesic as there is no effect on sham-operated animals (P>0.05). Although the CCI surgery is unilateral (left sciatic nerve), the allodynia is present bilaterally. In addition, the reversal of allodynia by A_{2A} agonism also occurs bilaterally. Therefore, ATL313 activates A_{2A} receptors within the spinal cord altering the mechanisms leading to central sensitization.

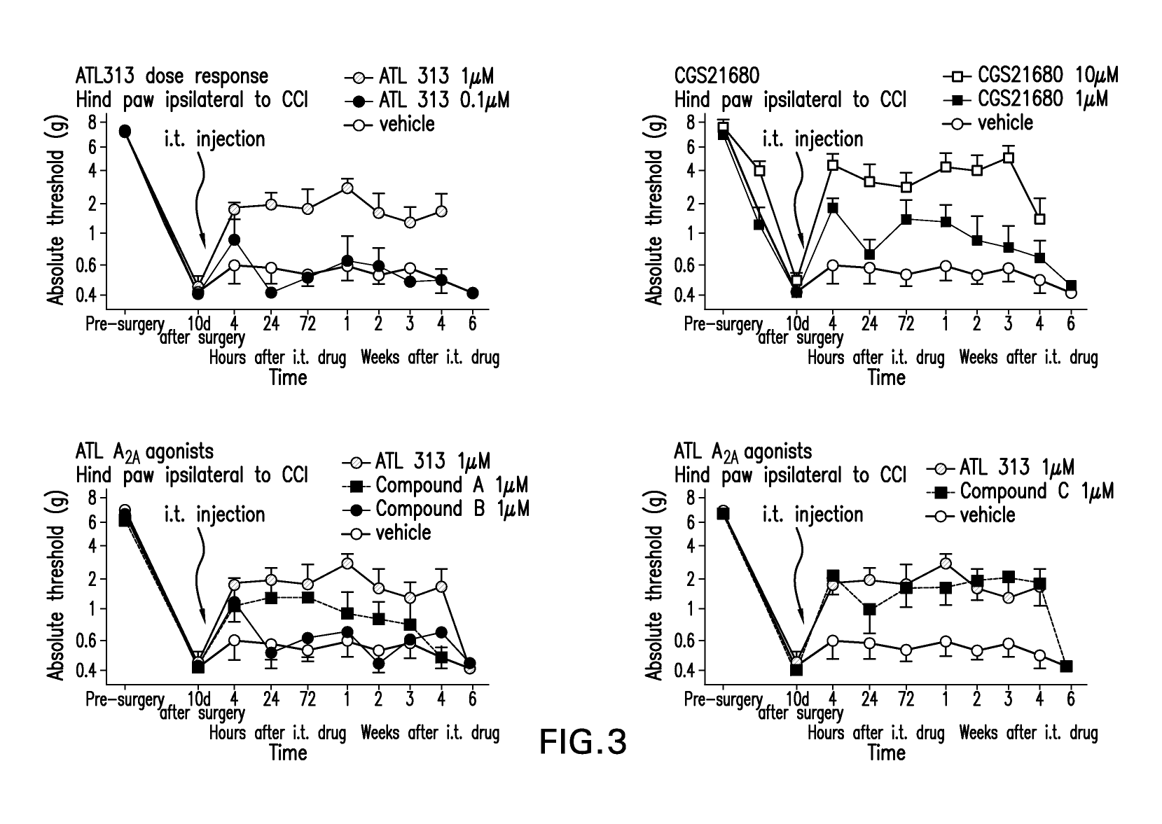

FIG. 3, top left panel, shows a dose-response of ATL313. The animals following unilateral CCI surgery of the left sciatic nerve, as noted above, have allodynia in both hind paws. For simplicity all graphs show left hind paw responses only, as the right hind paw had equivalent responses. A ten-fold lower dose of ATL313, 0.1 uM in 5 uL intrathecal administration had no significant impact on CCI-induced allodynia, as compared to saline-injected animals (P>0.05).

FIG. 3, top left panel, shows that CGS21680, a commercially available A_{2A} agonist (Sigma), produces a comparable reversal of CCI-induced allodynia, in both duration and intensity (P<0.001), but at a 10-fold higher dose than that of ATL313.

FIG. 3, bottom panels, show the effect of Compounds A, B, and C, which were tested at 1 uM. The results ranged between ATL313 (1 μM) and CGS21680 (1 μM). While the reason(s) for this variability in efficacy across A_{2A} agonists is at present unclear, some factors, which may potentially contribute to this variability, include binding efficacy and specificity, mobility and/or penetration of the drugs within the spinal cord.

Results:

A single intrathecal injection of an A_{2A} agonist can produce a remarkably enduring reversal of allodynia for at least four weeks. Duration of pain reversal was dose dependent, while peak magnitude of reversal was comparable across doses. Neither dose produced analgesia in sham-operated controls.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method for treating neuropathic pain, comprising: intrathecally administering to a patient in need thereof a therapeutically effective amount of an A_{2A} adenosine receptor agonist.

2. The method of claim 1, wherein the agonist is part of a pharmaceutical composition, further comprising: a pharmaceutically acceptable excipient.

3. The method of claim 1, wherein the agonist, comprises: a substituted 6-amino-9-(tetrahydrofuran-2′-yl)purine, or a pharmaceutically acceptable salt thereof.

4. The method of claim 1 wherein the agonist, comprises: a 6-amino-9-(3′,4′-dihydroxy-tetrahydrofuran-2′-yl)purine substituted at the 3- and 5′-positions, or a pharmaceutically acceptable salt thereof.

5. The method of claim 1 wherein the agonist, comprises: a 5-[6-amino-2-(3-piperidin-4-yl-prop-1-ynyl)-purin-9-yl]-3,4-dihydroxy-tetrahydro-furan-2-carboxylic acid cyclopropylamide, substituted on the piperidine nitrogen, or a pharmaceutically acceptable salt thereof.

6. The method of claim 1 wherein the agonist, comprises: a 4-{3-[6-amino-9-(5-cyclopropylcarbamoyl-3,4-dihydroxy-tetrahydro-furan-2-yl)-9H-purin-2-yl]-prop-2-ynyl}-piperidine-1-carboxylic acid ester or a pharmaceutically acceptable salt thereof.

7. The method of claim 1 wherein the agonist, comprises: a 5-[6-amino-2-(3-piperidin-4-yl-prop-1-ynyl)-purin-9-yl]-3,4-dihydroxy-tetrahydro-furan-2-carboxylic acid ethylamide, substituted on the piperidine nitrogen, or a pharmaceutically acceptable salt thereof.

8. The method of claim 1 wherein the agonist, comprises: a 4-{3-[6-amino-9-(5-ethylcarbamoyl-3,4-dihydroxy-tetrahydro-furan-2-yl)-9H-purin-2-yl]-prop-2-ynyl}-piperidine-1-carboxylic acid ester or a pharmaceutically acceptable salt thereof.

9. The method of claim 1, wherein the A_{2A} adenosine receptor agonist is a compound of formula I or a stereoisomer or pharmaceutically acceptable salt thereof: 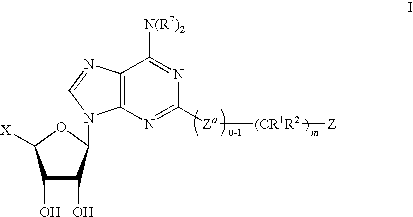
wherein
Z^{a} is C≡C, O, NH, or NHN═CR^{3a};
Z is CR³R⁴R⁵ or NR⁴R⁵;
each R¹ is independently hydrogen, halo, —OR^{a}, —SR^{a}, (C₁-C₈)alkyl, cyano, nitro, trifluoromethyl, trifluoromethoxy, (C₃-C₈)cycloalkyl, heterocycle, heterocycle(C₁-C₈)alkylene-, aryl, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, —OPO₃R^{a}, R^{a}OC(═S)—, R^{a}C(═S)—, —SSR^{a}, R^{a}S(═O)—, R^{a}S(═O)₂—, or —N═NR^{b};
each R² is independently hydrogen, halo, (C₁-C₈)alkyl, (C₃-C₈)cycloalkyl, heterocycle, heterocycle(C₁-C₈)alkylene-, aryl, aryl(C₁-C₈)alkylene-, heteroaryl, or heteroaryl(C₁-C₈)alkylene-;
alternatively, R¹ and R² and the atom to which they are attached is C═O, C═S or C═NR^{d},
R⁴ and R⁵ are independently H or (C₁-C₈)alkyl;
alternatively, R⁴ and R⁵ together with the atom to which they are attached form a saturated, partially unsaturated, or aromatic ring that is mono-, bi- or polycyclic and has 3, 4, 5, 6, 7, 8, 9 or 10 ring atoms optionally having 1, 2, 3, or 4 heteroatoms selected from non-peroxide oxy (—O—), thio (—S—), sulfinyl (—SO—), sulfonyl (—S(O)₂—) or amine (—NR^{b}—) in the ring;
wherein R⁴ and R⁵ are independently substituted with 0-3 R⁶ groups or any ring comprising R⁴ and R⁵ is substituted with from 0 to 6 R⁶ groups;
each R⁶ is independently hydrogen, halo, —OR^{a}, —SR^{a}, (C₁-C₈)alkyl, cyano, nitro, trifluoromethyl, trifluoromethoxy, (C₁-C₈)cycloalkyl, (C₆-C₁₂)bicycloalkyl, heterocycle, heterocycle (C₁-C₈)alkylene-, aryl, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, —OPO₃R^{a}, R^{a}OC(═S)—, R^{a}C(═S)—, —SSR^{a}, R^{a}S(═O)—, —NNR^{b}, or two R⁶ groups and the atom to which they are attached is C═O, C═S; or two R⁶ groups together with the atom or atoms to which they are attached can form a carbocyclic or heterocyclic ring comprising from 1-6 carbon atoms and 1, 2, 3, or 4 heteroatoms selected from non-peroxide oxy (—O—), thio (—S—), sulfinyl (—SO—), sulfonyl (—S(O)₂—) or amine (—NR^{b}—) in the ring;
R³ is hydrogen, halo, —OR^{a}, —SR^{a}, (C₁-C₈)alkyl, cyano, nitro, trifluoromethyl, trifluoromethoxy, (C₃-C₈)cycloalkyl, heterocycle, heterocycle(C₁-C₈)alkylene-, aryl, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, —OPO₃R^{a}, R^{a}OC(═S)—, R^{a}C(═S)—, —SSR^{a}, R^{a}S(═O)—, R^{a}S(═O)₂—, —NNR^{b}; or if the ring formed from CR⁴R⁵ is aryl or heteroaryl or partially unsaturated then R³ can be absent;
R^{3a} is hydrogen, (C₁-C₈)alkyl, or aryl;
each R⁷ is independently hydrogen, (C₁-C₈)alkyl, (C₃-C₈)cycloalkyl, aryl, aryl(C₁-C₈)alkylene, heteroaryl, or heteroaryl(C₁-C₈)alkylene-;
X is —CH₂OR^{a}, —CO₂R^{a}, —CH₂OC(O)R^{a}, —C(O)NR^{b}R^{c}, —CH₂SR^{a}, —C(S)OR^{a}, —CH₂OC(S)R^{a}, —C(S)NR^{b}R^{c}, or —CH₂N(R^{b})(R^{c});
alternatively, X is an aromatic ring of the formula: 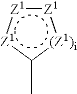
each Z¹ is non-peroxide oxy (—O—), S(O)₀₋₂, —C(R⁸)—, or amine (—NR⁸—), provided that at least one Z¹ is non-peroxide oxy (—O—), thio (—S—), sulfinyl (—SO—), sulfonyl (—S(O)₂—) or amine (—NR⁸—);
each R⁸ is independently hydrogen, (C₁-C₈)alkyl, (C₁-C₈)alkenyl, (C₃-C₈)cycloalkyl, (C₃-C₈)cycloalkyl(C₁-C₈)alkylene, (C₃-C₈)cycloalkenyl, (C₃-C₈)cycloalkenyl(C₁-C₈)alkylene, aryl, aryl(C₁-C₈)alkylene, heteroaryl, or heteroaryl(C₁-C₈)alkylene, wherein any of the alkyl or alkenyl groups of R⁸ are optionally interrupted by —O—, —S—, or —N(R^{a})—;
wherein any of the alkyl, cycloalkyl, heterocycle, aryl, or heteroaryl, groups of R¹, R², R³, R^{3a}, R⁶, R⁷ and R⁸ is optionally substituted on carbon with one or more (e.g. 1, 2, 3, or 4) substituents selected from the group consisting of halo, —OR^{a}, —SR^{a}, (C₁-C₈)alkyl, cyano, nitro, trifluoromethyl, trifluoromethoxy, (C₃-C₈)cycloalkyl, (C₆-C₁₂)bicycloalkyl, heterocycle, heterocycle(C₁-C₈)alkylene-, aryl, aryloxy, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, —OPO₃R^{a}, R^{a}OC(═S)—, R^{a}C(═S)—, —SSR^{a}, R^{a}S(═O)—, R^{b}R^{c}NS(O)ₚ, and —N═NR^{b};
wherein any (C₁-C₈)alkyl, (C₃-C₈)cycloalkyl, (C₆-C₁₂)bicycloalkyl, (C₁-C₈)alkoxy, (C₁-C₈)alkanoyl, (C₁-C₈)alkylene, or heterocycle, is optionally partially unsaturated;
each R^{a}, R^{b} and R^{c} is independently hydrogen, (C₁-C₁₂)alkyl, (C₁-C₈)alkoxy, (C₁-C₈)alkoxy-(C₁-C₁₂)alkylene, (C₃-C₈)cycloalkyl, (C₃-C₈)cycloalkyl-(C₁-C₁₂)alkylene, (C₁-C₈)alkylthio, amino acid, aryl, aryl(C₁-C₈)alkylene, heterocycle, heterocycle-(C₁-C₈)alkylene, heteroaryl, or heteroaryl(C₁-C₈)alkylene;
alternatively R^{b} and R^{c}, together with the nitrogen to which they are attached, form a pyrrolidino, piperidino, morpholino, or thiomorpholino ring;
wherein any of the alkyl, cycloalkyl, heterocycle, aryl, or heteroaryl groups of R^{a}, R^{b} and R^{c} is optionally substituted on carbon with 1 or 2 substituents selected from the group consisting of halo, —(CH₂)ₐOR^{e}, —(CH₂)ₐSR^{e}, (C₁-C₈)alkyl, (CH₂)ₐCN, (CH₂)ₐNO₂,
trifluoromethyl, trifluoromethoxy, —(CH₂)ₐCO₂R³, (CH₂)ₐNR^{e}R^{e}, and (CH₂)ₐC(O)NR^{e}R^{e};
R^{d} is hydrogen or (C₁-C₆)alkyl;
R^{e} is independently selected from H and (C₁-C₆)alkyl;
a is 0, 1, or 2;
i is 1 or 2
m is 0 to 8; and
p is 0 to 2;
provided that m is at least 1 when Z is NR⁴R⁵; or
a pharmaceutically acceptable salt thereof.

10. The method of claim 9, wherein the A_{2A} adenosine receptor agonist is a compound selected from the compounds of the following table or a stereoisomer or pharmaceutically acceptable salt thereof:

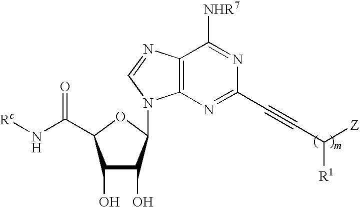

| Ex. | | | |
|---|---|---|---|
| # | R^{c} | R⁷ | —(R¹)ₘ-Z |
| 1. | Et | H | 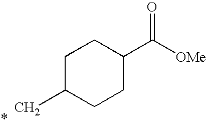 |
| 2. | Et | H | 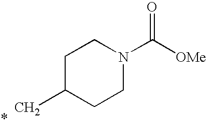 |
| 3. | cPr | H | 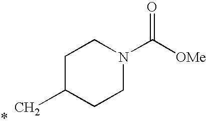 |
| 4. | Et | H | 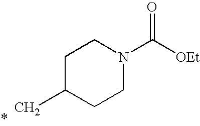 |
| 5. | cPr | H | 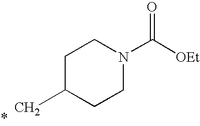 |
| 6. | Et | H | 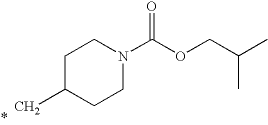 |
| 7. | cPr | H | 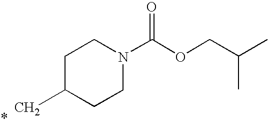 |
| 8. | Et | H | 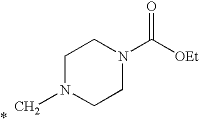 |
| 9. | Et | H | 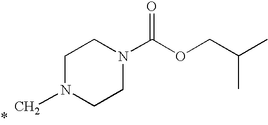 |
| 10. | Et | H | 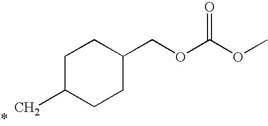 |
| 11. | Et | H | 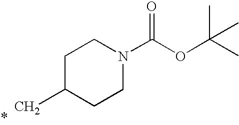 |
| 12. | cPr | H | 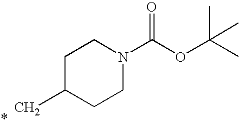 |
| 13. | Et | H | 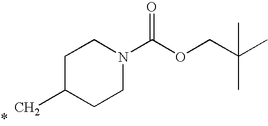 |
| 14. | cPr | H | 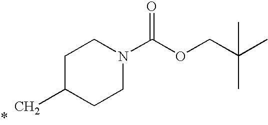 |
| 15. | Et | H | 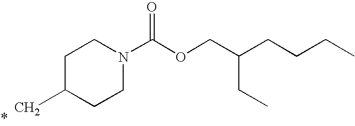 |
| 16. | cPr | H | 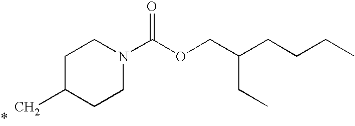 |
| 17. | cPr | H | 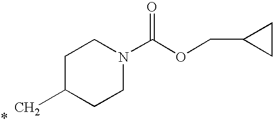 |
| 18. | Et | H | 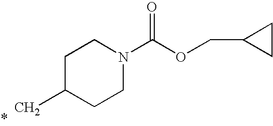 |
| 19. | cPr | H | 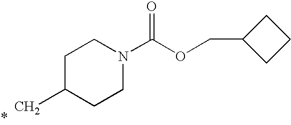 |
| 20. | Et | H | 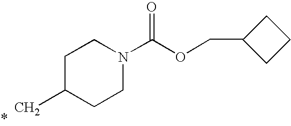 |
| 21. | cPr | H | 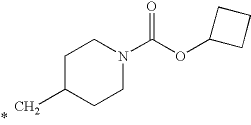 |
| 22. | Et | H | 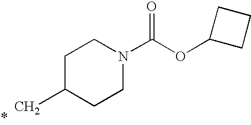 |
| 23. | Et | H | 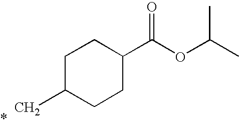 |
| 24. | cPr | H | 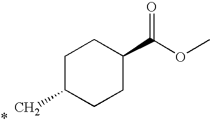 |
| 25. | Et | H | 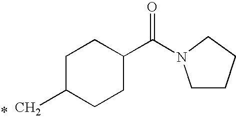 |
| 26. | Et | H | 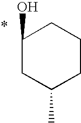 |
| 27. | Et | H | 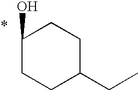 |
| 28. | Et | H | 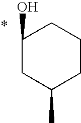 |
| 29. | Et | H | 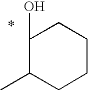 |
| 30. | Et | H | 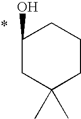 |
| 31. | cPr | H | 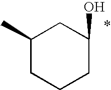 |
| 32. | Et | H | 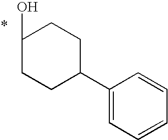 |
| 33. | Et | H | 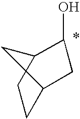 |
| 34. | cPr | H | 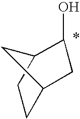 |
| 35. | cPr | H | 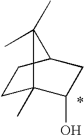 |
| 36. | Et | H | 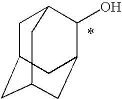 |
| 37. | cPr | H | 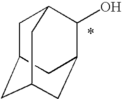 |
| 38. | Et | H | 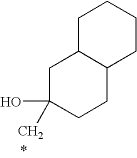 |
| 39. | cPr | H | 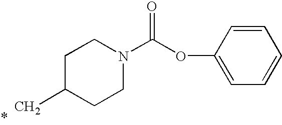 |
| 40. | Et | H | 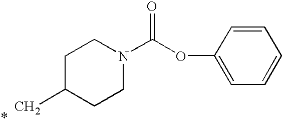 |
| 41. | cPr | H | 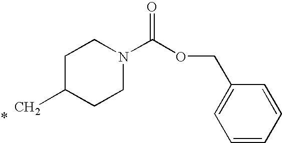 |
| 42. | Et | H | 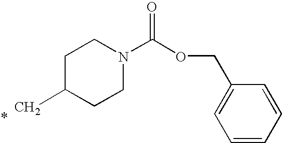 |

* signifies the point of attachment.

11. The method of claim 1, wherein the A_{2A} adenosine receptor agonist is a compound of formula TI or a stereoisomer or pharmaceutically acceptable salt thereof: 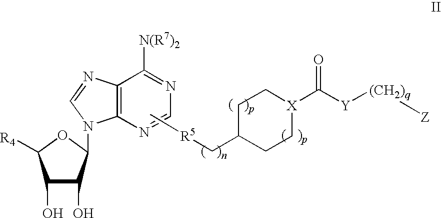
wherein:
R¹ and R² independently are selected from the group consisting of H, (C₁-C₈)alkyl, (C₃-C₈)cycloalkyl, (C₃-C₈)cycloalkyl(C₁-C₈)alkylene, aryl, aryl(C₁-C₈)alkylene, heteroaryl, heteroaryl(C₁-C₈)alkylene-, diaryl(C₁-C₈)alkylene, and diheteroaryl(C₁-C₈)alkylene, wherein the aryl and heteroaryl rings are optionally substituted with 1-4 groups independently selected from fluoro, chloro, iodo, bromo, methyl, trifluoromethyl, and methoxy;
each R independently is selected from the group consisting of H, C₁-C₄ alkyl, cyclopropyl, cyclobutyl, and (CH₂)ₐcyclopropyl;
X is CH or N, provided that when X is CH then Z cannot be substituted with halogen, C₁-C₆ alkyl, hydroxyl, amino, or mono- or di-(C₁-C₆-alkyl)amino;
Y is selected from the group consisting of O, NR¹, —(OCH₂CH₂O)ₘCH₂—, and —(NR¹CH₂CH₂O)ₘCH₂—, provided that when Y is O or NR¹, then at least one substituent is present on Z;
Z is selected from the group consisting of 5-membered heteroaryl, 6-membered aryl, 6-membered heteroaryl, carbocyclic biaryl, and heterocyclic biaryl, wherein the point of attachment of Y to Z is a carbon atom on Z, wherein Z is substituted with 0-4 groups independently selected from the group consisting of F, Cl, Br, I, (C₁-C₄)alkyl, —(CH₂)ₐOR³, —(CH₂)ₐNR³R³, —NHOH, —NR³NR³R³, nitro, —(CH₂)ₐCN, —(CH₂)ₐCO₂R³, —(CH₂)ₐCONR³R³, trifluoromethyl, and trifluoromethoxy;
alternatively, Y and Z together form an indolyl, indolinyl, isoindolinyl, tetrahydroisoquinolinyl, or tetrahydroquinolinyl moiety wherein the point of attachment is via the ring nitrogen and wherein said indolyl, indolinyl, isoindolinyl, tetrahydroisoquinolinyl, or tetrahydroquinolinyl moiety, which is substituted with 0-4 groups independently selected from the group consisting of F, Cl, Br, I, C₁-C₄ alkyl, —(CH₂)ₐOR³, —(CH₂)ₐNR³R³, —NHOH, —NR³NR³R³, NO₂, —(CH₂)ₐCN, —(CH₂)ₐCO₂R³, —(CH₂)ₐCONR³R³, CF₃, and OCF₃;
R³ is independently selected from the group consisting of H, (C₁-C₆)alkyl, cycloalkyl, aryl, and heteroaryl;
R⁴ is selected from the group consisting of CH₂OR, C(O)NRR, and CO₂R;
R⁵ is selected from the group consisting of CH₂CH₂, CH═CH, and C≡C;
a is selected from 0, 1, and 2;
m is selected from 1, 2, and 3;
n is selected from 0, 1, and 2;
each p independently is selected from 0, 1, and 2; and,
q is selected from 0, 1, and 2.

12. The method of claim 11, wherein the A_{2A} adenosine receptor agonist is a compound selected from the compounds of the following table or a stereoisomer or pharmaceutically acceptable salt thereof:

| | |
|---|---|
| | i |
| 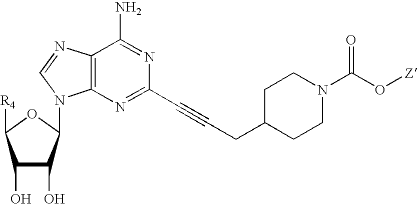 | |
| | |
|---|---|
| | ii |
| 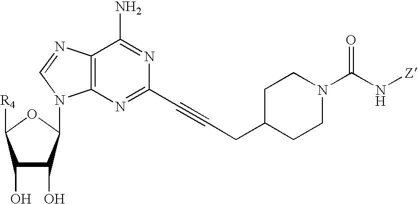 | |
| | iii |
| 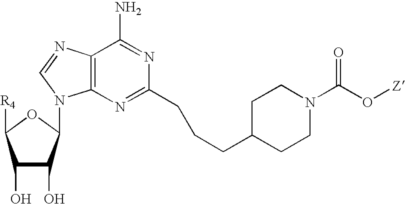 | |
| Ex. # | R⁴ | Z′ |
|---|---|---|
| 1 | C | 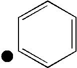 |
| 2 | C | 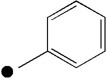 |
| 3 | C | 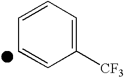 |
| 4 | A | 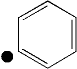 |
| 5 | C | 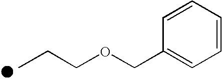 |
| 6 | A | 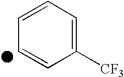 |
| 7 | A | 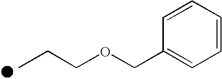 |
| 8 | C | 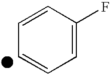 |
| 9 | C | 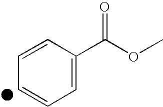 |
| 10 | C | 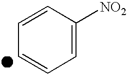 |
| 11 | A | 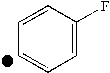 |
| 12 | A | 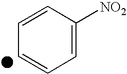 |
| 13 | A | 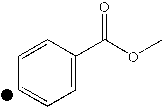 |
| 14 | C | 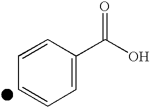 |
| 15 | B | 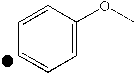 |
| 16 | B | 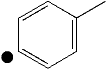 |
| 17 | C | 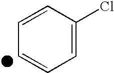 |
| 18 | C | 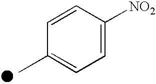 |
| 19 | B | 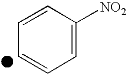 |
| 20 | C | 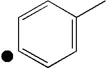 |
| 21 | C | 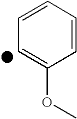 |
| 22 | C | 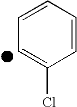 |
| 23 | C | 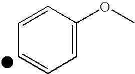 |
| 24 | B | 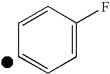 |
| 25 | B | 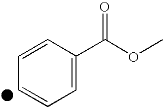 |
| 26 | B | 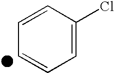 |
| 27 | A | 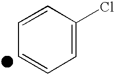 |
| 28 | A | 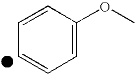 |
| 29 | A | 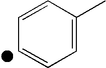 |
| 30 | A | 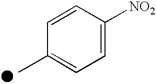 |
| 31 | B | 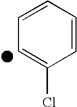 |
| 32 | B | 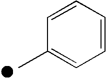 |
| 33 | B | 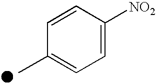 |
| 34 | B | 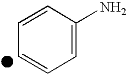 |
| 35 | A | 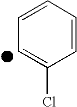 |
| 36 | A | 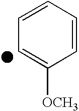 |
| 37 (iii) | B | 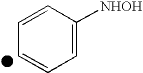 |
| 38 (iii) | C | 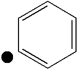 |
| 39 (iii) | C | 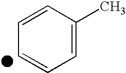 |
| 40 (iii) | C | 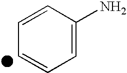 |
| 41 (iii) | C | 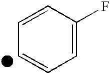 |
| 42 | C | 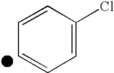 |
| 43 (ii) | C | 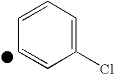 |
| 44 (ii) | A | 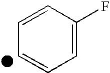 |
| 45 (ii) | A | 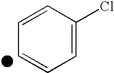 |
| 46 (ii) | A | 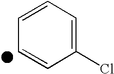 |
| 47 (ii) | C | 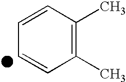 |
| 48 (ii) | C | 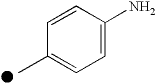 |
| 49 | B | 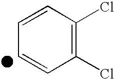 |
| 50 | B | 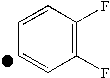 |
| 51 | C | 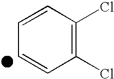 |
| 52 | C | 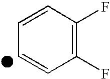 |
| 53 | A | 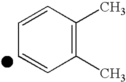 |
| 54 | A | 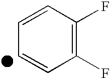 |
| 55 | A | 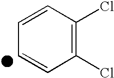 |
| 56 | C | 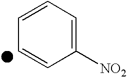 |
| 57 | C | 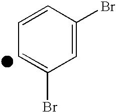 |

R⁴ = A: CH₂OH; B: C(O)NEthyl; C: C(O)NCyclopropyl;
Compounds are of formula (i), unless indicated.

13. The method of claim 1, wherein the A_{2A} adenosine receptor agonist is a compound of formula (Ib)-(Id) or a pharmaceutically acceptable salt thereof:

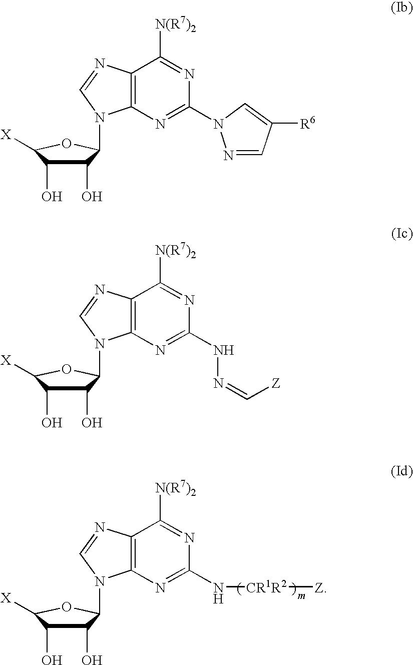

14. The method of claim 1, wherein the A_{2A} adenosine receptor agonist is selected from: 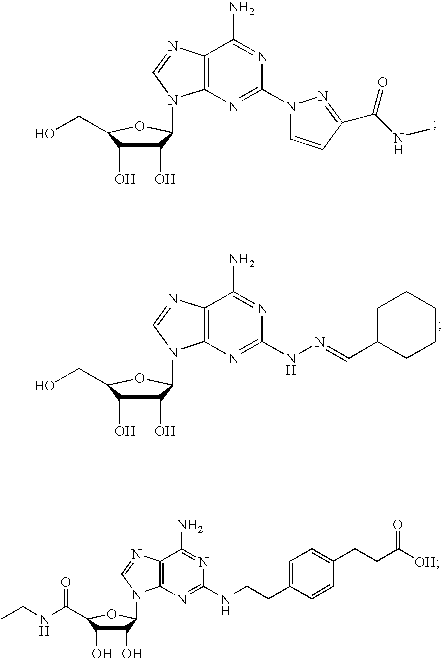
or a pharmaceutically acceptable salt thereof.

15. The method of claim 1, wherein the A_{2A} adenosine receptor agonist is a compound of the following formula or a pharmaceutically acceptable salt thereof:

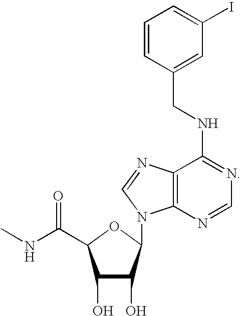

16. The method of claim 1, wherein the A_{2A} adenosine receptor agonist is a compound of the following formula or a pharmaceutically acceptable salt thereof: [chemical structure]
